(19) United States
(12) Patent Application Publication
(10) Pub. No.: US 2025/0376471 A1
(43) Pub. Date: Dec. 11, 2025

(54) CRYSTALLINE FORMS OF 2-[3-[4-AMINO-3-(2-FLUORO-4-PHENOXY-PHENYL)PYRAZOLO[3,4-D]PYRIMIDIN-1-YL]PIPERIDINE-1-CARBONYL]-4-METHYL-4-[4-(OXETAN-3-YL)PIPERAZIN-1-YL]PENT-2-ENENITRILE

(71) Applicant: Principia Biopharma Inc., Bridgewater, NJ (US)

(72) Inventors: Kolbot By, San Ramon, CA (US); Pasit Phiasivongsa, Hillsborough, CA (US); Carim Van Beek, Foster City, CA (US)

(73) Assignee: Principia Biopharma Inc., Bridgewater, NJ (US)

(21) Appl. No.: 18/874,401

(22) PCT Filed: Jun. 13, 2023
(86) PCT No.: PCT/US2023/025124

§ 371 Date: Dec. 12, 2024

Related U.S. Application Data
(60) Provisional application No. 63/352,027, filed on Jun. 14, 2022.

(51) Int. Cl. C07D 487/04; A61K 31/519

(52) U.S. Cl. CPC C07D 487/04; A61K 31/519

(57) ABSTRACT

Provided herein are substantially crystalline solid forms of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pen-t-2-enenitrile (identified herein as Compound (I), and also known as PRN 1008 or rilzabrutinib) as a pharmaceutically-acceptable salt selected from an HCI, oxalate, and/or a maleate salt or alternatively, as a pharmaceutically-acceptable methyl paraben co-crystal, and pharmaceutical compositions comprising the substantially crystalline forms.

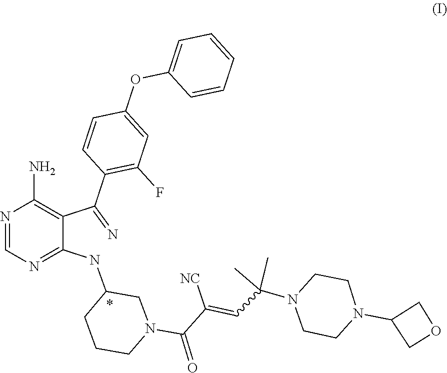 [SMILES: CC(C)(C=C(C#N)C(=O)N1CCCC(Nc2ncnc(N)c2C(=N)c2ccc(Oc3ccccc3)cc2F)C1)N1CCN(C2COC2)CC1]

### FIELD

The present disclosure relates to solid forms of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (identified herein as Compound (I) and also known as PRN 1008 or rilzabrutinib), which is a potent inhibitor of the Bruton's Tyrosine Kinase (“BTK”) useful in the treatment of cancer and other conditions including autoimmune diseases. The present disclosure further relates to pharmaceutical compositions comprising said forms.

### BACKGROUND

The enzyme BTK is a member of the Tec family of non-receptor tyrosine kinases. BTK is expressed in most hematopoietic cells including B cells, mast cells, and macrophages. BTK plays a role in the development and activation of B cells. BTK activity has been implicated in the pathogenesis of several disorders and conditions including B cell-related hematological cancers (such as non-Hodgkin lymphoma and B cell chronic lymphocytic leukemia) and autoimmune diseases (such as immune thrombocytopenia (ITP), rheumatoid arthritis, Sjogren's syndrome, pemphigus, inflammatory bowel disease (IBD), lupus nephritis, atopic dermatitis, warm autoimmune hemolytic anemia, asthma and other acute respiratory distress, and chronic spontaneous urticaria).

Accordingly, pharmaceutical compositions comprising therapeutically-effective amounts of BTK inhibitors may be useful in the treatment of certain cancers and autoimmune diseases.

When treating certain cancers and autoimmune diseases with BTK inhibitors, it is also desirable that the treatment can be administered in a form that is easily absorbed by the body and also shelf stable. The pharmaceutically active substance used to prepare the treatment should be as pure as possible and its stability on long-term storage should be guaranteed under various environmental conditions. These properties are useful to prevent the appearance of unintended degradation products in pharmaceutical compositions, which degradation products may be potentially toxic or result simply in reducing the potency of the composition.

Further, a frequent concern for the large-scale manufacture of pharmaceutical compounds is that the active substance has a crystalline morphology to ensure consistent processing parameters and pharmaceutical quality. In this regard, changes to the solid state of a pharmaceutical composition which improve its physical and chemical stability may give a significant advantage over less stable forms of the same drug.

When a compound crystallizes from a solution or slurry, it may crystallize with different spatial lattice arrangements, a property referred to as “polymorphism.” Each of the crystal forms is a “polymorph.” Although polymorphs of a given substance have the same chemical composition, they may differ from each other with respect to one or more physical properties, such as solubility, dissociation, true density, dissolution, melting point, crystal shape, compaction behavior, flow properties, and/or solid-state stability. Thus, different polymorphs may present significant advantages, or disadvantages, in the preparation of pharmaceutical compositions.

Compound (I) is a BTK inhibitor having the following structure:

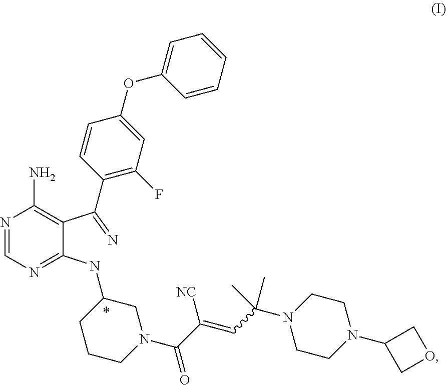 [SMILES: CC(C)(C=C(C#N)C(=O)N1CCCC(Nc2ncnc(N)c2C(=N)c2ccc(Oc3ccccc3)cc2F)C1)N1CCN(C2COC2)CC1]

where *C is a stereochemical center. Compound (I) is preferably present substantially as the (R)-enantiomer, which is also known as PRN 1008 or alternatively, as rilzabrutinib.

Compound (I) is disclosed and claimed in U.S. Pat. No. 9,266,895 (Example 31 thereof) (corresponding to WO 2014/039899). The procedures described in U.S. Pat. No. 9,266,895 provide Compound (I) as a white amorphous solid following solvent extraction wherein residual solvents are present in levels above limits suitable for preparation of pharmaceutical compositions.

Alternative procedures for producing Compound (I) and solid forms thereof are disclosed in WO 2015/127310 and U.S. Pub. No. 2021/0198264. The '264 publication discloses processes for preparing Compound (I) that produce amorphous forms of the compound with pharmaceutically-acceptable levels of residual solvents remaining (e.g., methanol, isopropyl acetate, and heptane). In some embodiments described in the '264 publication, no detectible levels of such residual solvents remain in the final product.

Certain crystalline forms of Compound (1) are disclosed in US 2021/0221818 (corresponding to WO2021/150723), including crystalline forms denoted therein as Form A, Form B and Form C.

Producing a pharmaceutical composition for the effective treatment of disease also requires, however, that the compound can be produced in a form that is shelf-stable and easily formulated into a composition that may be readily absorbed by the body via not only immediate absorption but with modified and extended-release formulations. The pharmaceutically active substance used to prepare the treatment should be as pure as possible and maintain its stability over long-term storage under various environmental conditions. These properties are useful to prevent the formation of degradation products in the pharmaceutical compositions that may potentially be toxic or result simply in reducing the potency of the composition.

If an unstable crystalline form is used, crystal morphology may change during manufacture and/or storage, resulting in quality control problems and formulation irregularities. Such a change may affect the reproducibility of the manufacturing process and thus lead to final formulations which do not meet the high quality and stringent requirements imposed on formulations of pharmaceutical compositions.

Accordingly, there remains a need for crystalline forms of Compound (I) having sufficient stability and capability for bio-absorption to be suitable for preparing effective pharmaceutical formulations and treatments including compositions designed for extended and modified release.

### SUMMARY

The present disclosure relates to new solid forms of Compound (I) that offer some surprisingly improved solubility and stability as compared to Compound (I), Form B disclosed in US 2021/0221818 (corresponding to WO2021/150723), or as compared to other solid, salt or crystalline forms of Compound (I) previously disclosed.

Disclosed herein are substantially crystalline forms of Compound (I), chosen from HCl salt forms, oxalate salt forms, and maleate salt forms.

Also disclosed herein is a co-crystalline form of Compound (I), optionally, a substantially crystalline form of Compound (I) as a co-crystal comprising substantially crystalline Compound (I) and a coformer, in one embodiment where the coformer is methyl paraben.

The present disclosure also relates to a pharmaceutical composition comprising at least one solid form of Compound (I) as described herein and a pharmaceutically acceptable excipient.

The present disclosure further relates to pharmaceutical compositions comprising substantially crystalline forms of Compound (I) disclosed herein formulated with at least one excipient selected from fillers, drug release modifiers, disintegrants, and lubricants to provide a solid dosage formulation to administer to a subject in need of treatment that provides improved solubility, stability, and/or bio-absorption as compared to Compound (I), Form B disclosed in US 2021/0221818 (corresponding to WO2021/150723).

The present disclosure still further relates to pharmaceutical compositions comprising substantially crystalline forms of Compound (I) as described herein.

Also disclosed herein are methods of treating a disease in a subject that is mediated by activity of BTK by administering to the subject a pharmaceutical composition comprising at least one substantially crystalline form of Compound (I) as described herein.

The accompanying drawings, briefly described below, are incorporated herein and constitute a part of this specification. The drawings illustrate several embodiments of the present disclosure and should be considered together with the description.

### BRIEF DESCRIPTION OF THE DRAWINGS

FIG. 1 shows an overlay of XRPD patterns of two different birefringent crystalline forms of Compound (I): HCl crystalline salt, obtained using CuKα radiation.

FIG. 2 shows an XRPD pattern of Compound (I): HCl crystalline salt hydrate obtained using CuKα radiation.

FIG. 3 shows DSC/TGA thermograms of Compound (I): HCl crystalline salt hydrate.

FIG. 4 shows an XRPD pattern of Compound (I): HCl crystalline salt Form A, obtained using CuKα radiation.

FIG. 5 shows an additional XRPD pattern of Compound (I): HCl crystalline salt Form A, with peaks numbered, obtained using CuKα radiation.

FIG. 6 shows a TGA thermogram of Compound (I): HCl crystalline salt Form A.

FIG. 7 shows DSC/TGA thermograms of Compound (I): HCl crystalline salt Form A.

FIG. 8 shows an XRPD pattern of Compound (I): oxalate birefringent crystalline salt hydrate Form A, obtained using CuKα radiation.

FIG. 9 shows DSC/TGA thermograms of Compound (I): oxalate birefringent crystalline salt hydrate Form A.

FIG. 10 shows an XRPD pattern of Compound (I): maleate birefringent crystalline salt, obtained using CuKα radiation.

FIG. 11 shows DSC/TGA thermograms of Compound (I): maleate birefringent crystalline salt.

FIG. 12 shows an XRPD pattern of Compound (I) oxalate crystalline salt Form B, obtained using CuKα radiation.

FIG. 13 shows DSC/TGA thermograms of Compound (I) oxalate crystalline salt Form B.

FIG. 14 shows an overlay of XRPD patterns of Compound (I) oxalate crystalline salt hydrate Form A and Compound (I) oxalate crystalline salt hydrate Form B, obtained using CuKα radiation.

FIGS. 15A and 15B show the DSC thermograms of Compound (I) oxalate crystalline salt Form A (15A) and Form B (15B).

FIG. 16 shows an XRPD pattern of Compound (I) oxalate crystalline salt hydrate Form A, obtained using CuKα radiation.

FIG. 17 shows an additional XRPD pattern of Compound (I) oxalate crystalline salt hydrate Form A, with peaks numbered, obtained using CuKα radiation.

FIG. 18 shows a TGA thermogram of Compound (I) oxalate crystalline salt hydrate Form A.

FIG. 19 shows DSC/TGA thermograms of Compound (I) oxalate crystalline salt Form A.

FIG. 20 shows an XRPD pattern of Compound (I): maleate crystalline salt Form B (MeCN solvate), obtained using CuKα radiation.

FIG. 21 shows DSC/TGA thermograms of Compound (I): maleate crystalline salt Form B (MeCN solvate).

FIG. 22 shows an overlay of XRPD patterns of Compound (I): maleate crystalline salt Form A, maleic acid, and Compound (I) Form B disclosed in US 2021/0221818 (corresponding to WO2021/150723), obtained using CuKα radiation.

FIG. 23 shows a TGA thermogram of Compound (I): maleate crystalline salt Form A.

FIGS. 24A-C show comparative solubility assessment data for HCl crystalline salt of Compound (I) (FIG. 24A), crystalline Compound (I) Form B disclosed in US 2021/0221818 (corresponding to WO2021/150723) (FIG. 24B), and Compound (I) amorphous Form B (FIG. 24C).

FIG. 25 shows data in graphic form demonstrating the solubility of Compound (I) oxalate crystalline salt Form A over time in different pH.

FIG. 26 shows data in graphic form demonstrating the solubility of Compound (I) maleate crystalline salt over time in different pH.

FIG. 27 shows an XRPD pattern of Compound (I): methyl paraben co-crystal, obtained using CuKα radiation.

FIG. 28 shows a DSC/TGA thermogram of Compound (I): methyl paraben co-crystal.

FIG. 29 shows XRPD patterns of Compound (I): methyl paraben co-crystal, obtained using CuKα radiation, collected pre- and post-grinding as described in Example 11.

FIG. 30 shows XRPD patterns of Compound (I) Form B disclosed in US 2021/0221818 (corresponding to WO2021/150723), obtained using CuKα radiation, collected pre- and post-grinding as described in Example 11.

FIG. 31 shows XRPD patterns of Compound (I): methyl paraben co-crystal, obtained using CuKα radiation, collected pre- and post-compression as described in Example 12.

FIG. 32 shows XRPD patterns of Compound (I): methyl paraben co-crystal, obtained using CuKα radiation, collected pre- and post-storage as described in Example 13.

### DETAILED DESCRIPTION

Reference will now be made in detail to certain embodiments illustrated in the following Examples and accompanying drawings. While the disclosure provides exemplary embodiments, it will be understood that the examples are not intended to limit the disclosure to those embodiments. On the contrary, the disclosure is intended to cover all alternatives, modifications, and equivalents which may be appreciated by one skilled in the field from the disclosures.

Any section headings used herein are for organizational purposes only and are not to be construed as limiting the desired subject matter in any way.

### I. Definitions

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

“About” as used herein preceding one or more specific numerical values is intended to denote that a range of values are encompassed within the specific value to the extent one of ordinary skill in the art would consider the range equivalent to the specific, recited value (e.g., having the same function or result). When “about” precedes a list of numerical values or ranges, the term modifies all the values or ranges provided in the list.

For example, “about” when used herein with reference to peak points of an XRPD pattern denotes that the numerical value of the peak point may be within ±0.2 of the specific number indicated. As one skilled in the field will appreciate, the specific XRPD peaks are not precise but may vary in either direction with the experimental conditions including the size and purity of the sample.

As used herein, “birefringent” refers to a crystal that has two indices of refraction. Birefringence is also known as double refraction, and it occurs in anisotropic crystalline forms.

“Co-crystal,” “co-crystal of Compound (I),” or “Compound (I) co-crystal” as used herein means that Compound (I) is present in crystalline form and non-covalently bonded in a crystal lattice in a stoichiometric ratio with at least one coformer.

“Coformer” means the compound, or compounds, other than Compound (I) in the crystal lattice comprising the co-crystal. For example, with respect to the co-crystals of Compound (I) made herein, the coformer is the molecule in the co-crystal other than Compound (I), for example, methyl paraben. The co-crystal may also contain stoichiometric amounts of water compared to the Compound (I) and methyl paraben such as with a monohydrate or a dihydrate.

Compound (I) as used herein means 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, having the structure:

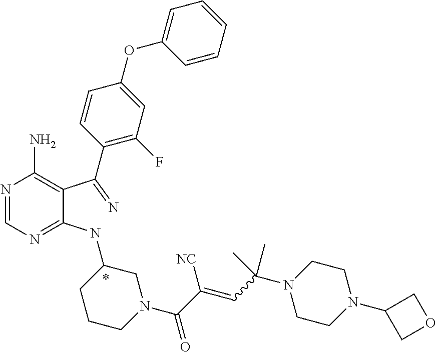 [SMILES: CC(C)(C=C(C#N)C(=O)N1CCCC(Nc2ncnc(N)c2C(=N)c2ccc(Oc3ccccc3)cc2F)C1)N1CCN(C2COC2)CC1]

Compound (I) as used herein denotes all stereoisomers or enantiomers thereof, as well as all mixtures of stereoisomers and enantiomers. Herein, Compound (I) may be referred to as a “drug,” “active agent,” “a therapeutically active agent,” or a “API.”

“Form B,” “Form B of Compound (I),” or “Compound (I) Form B” as used herein refers to a crystalline form of Compound (I) described as Form B in US 2021/0221818 (corresponding to WO2021/150723), which one skilled in the field will appreciate may be confirmed with characterization data and/or through the procedures disclosed in the referenced publication. “Form B” disclosed in US 2021/0221818 (corresponding to WO2021/150723) is a crystalline form that is distinct from Form B of the oxalate salt and/or Form B of the maleate salt that are disclosed herein.

“Substantially crystalline” or “substantially crystalline form” means that crystalline form(s) of compound(s) (including mixtures of crystalline compounds) in a sample are present in an amount of more than 50% by weight as compared with amorphous form(s) of compounds in a sample, optionally at least 60% or more, or by at least 70% or more by weight which can be readily measured with analytical tools available to one skilled in the art.

“Substantially pure” or “substantially pure crystalline” means that a single crystalline form of compound is present in a sample in an amount of 90% or more by weight as compared with all other ingredients in a sample including all other crystalline or amorphous forms of compounds and/or residual solvents or excipients; optionally, in an amount of 95% or more, also optionally, in an amount of 99% or more, as compared with all other ingredients.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use.

As used herein, the terms “room temperature” or “ambient conditions” refers to room temperature, open air, and uncontrolled humidity conditions typically at a temperature ranging from about 15° C. to about 30° C.

As used herein, the term “inhibit,” “inhibition,” or ‘inhibiting” refers to the reduction or suppression of a given condition, symptom, or disorder, or disease, or a significant decrease in the baseline activity of a biological activity or process.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As used herein, the term “solid form” refers to a physical form of a compound that is not predominantly in a liquid or gaseous state, including amorphous and crystalline forms.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. For example, an amorphous material is a solid material having no sharp characteristic signal(s) in its X-ray power diffractogram (i.e., is not crystalline as determined by XRPD). Instead, one or more broad peaks (e.g., halos) appear in its diffractogram. Broad peaks are characteristic of an amorphous solid. See, e.g., US 2004/0006237 for a comparison of diffractograms of an amorphous material and a crystalline material.

As used herein, the term “DSC” refers to the analytical method of differential scanning calorimetry.

As used herein, the term “TGA” refers to the analytical method of thermogravimetric (also referred to as thermogravimetric) analysis.

### II. Embodiments

The present disclosure relates to new solid forms of Compound (I) that offer surprisingly improved solubility and/or stability as compared to Compound (I), Form B disclosed in US 2021/0221818 (corresponding to WO2021/150723), or as compared to other solid, salt or crystalline forms of Compound (I) previously disclosed.

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile chosen from an HCl salt, oxalate salt, or maleate salt form thereof.

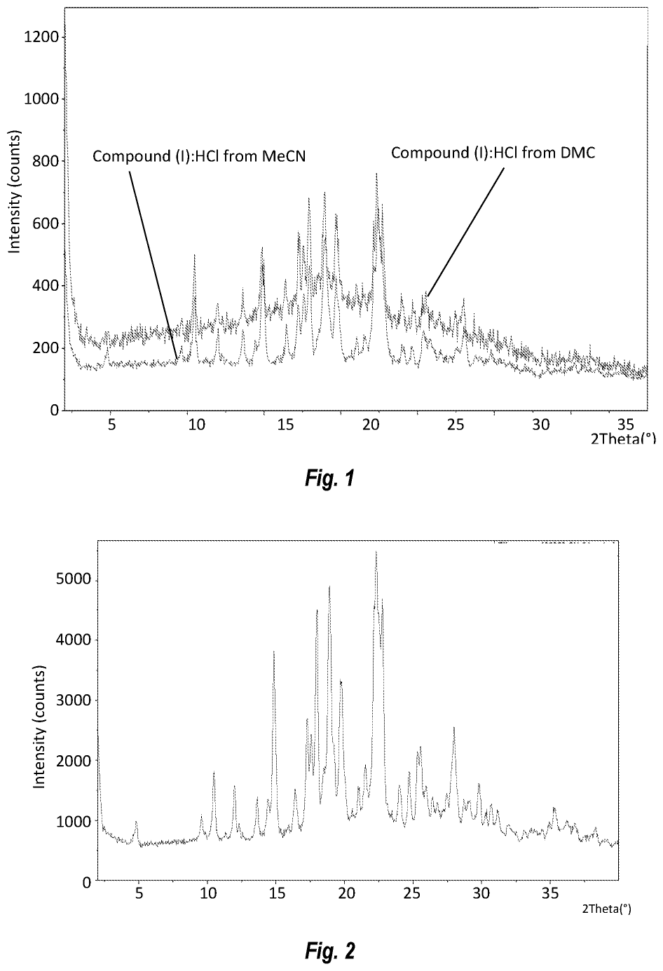

In some embodiments, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl birefringent crystalline salt. In some embodiments, the aforesaid HCl birefringent crystalline salt is characterized by an XRPD pattern substantially the same as those in FIG. 1 herein.

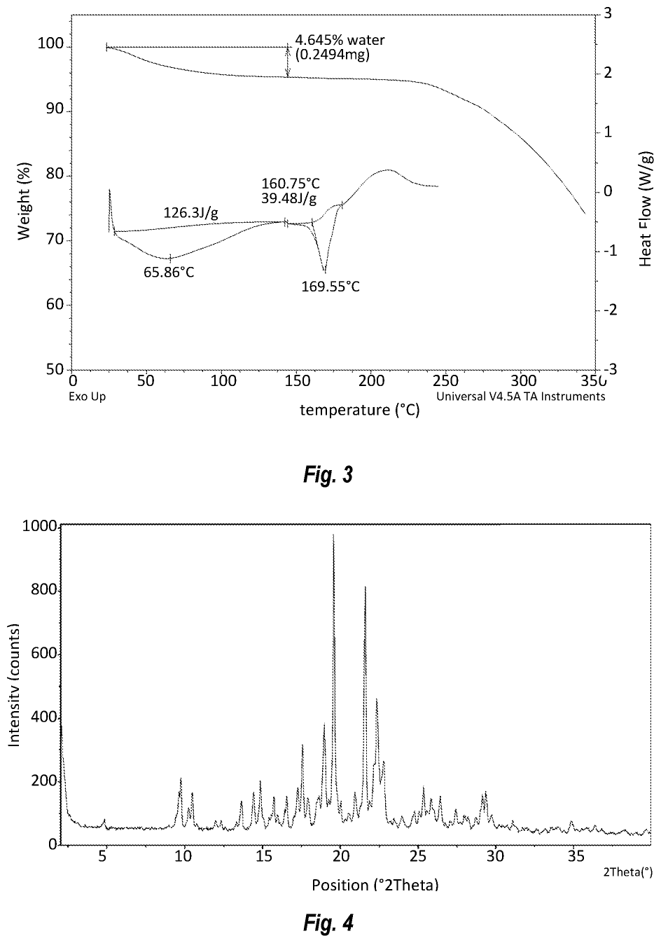

In some embodiments, the substantially crystalline form of the disclosure is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt hydrate. In some embodiments, the aforesaid HCl crystalline salt hydrate is characterized by at least one of an XRPD pattern substantially the same as FIG. 2 herein; and/or a DSC/TGA profile substantially the same as FIG. 3 herein.

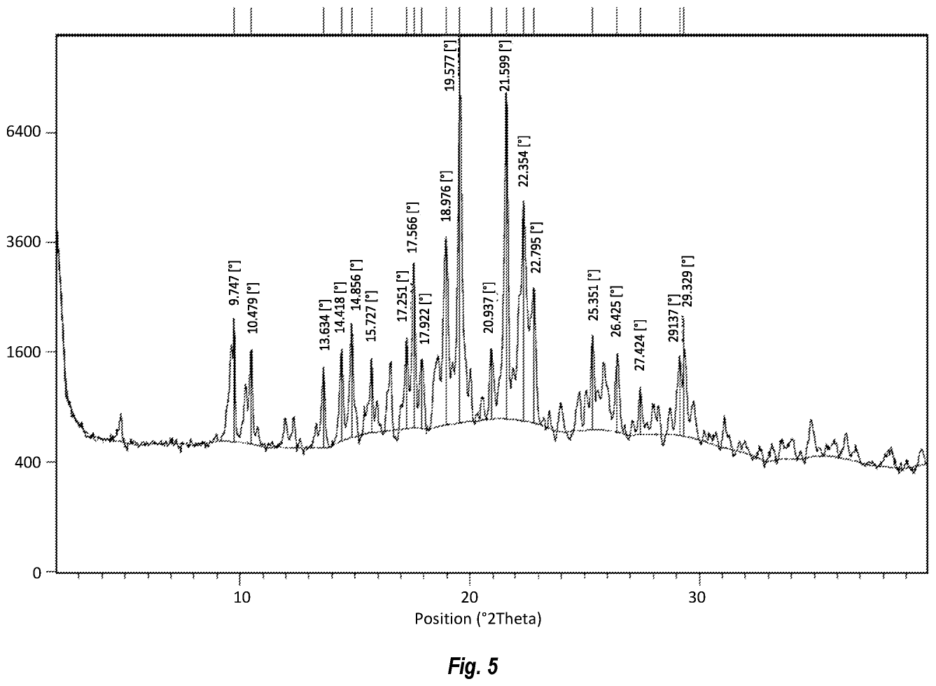

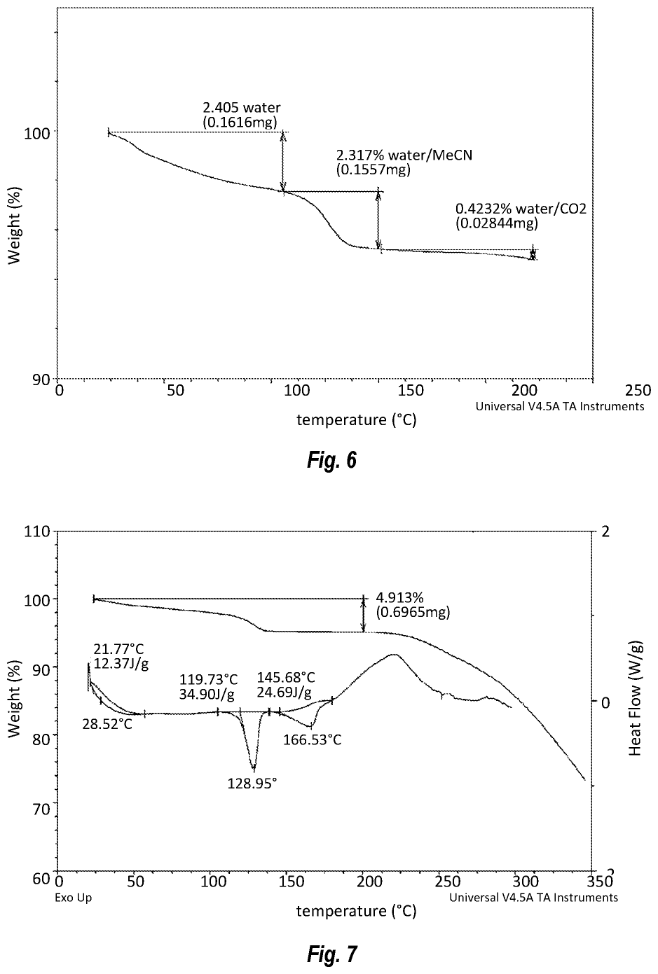

In some embodiments, the substantially crystalline form of the disclosure is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt Form A. In some embodiments, the aforesaid HCl crystalline salt Form A is characterized by at least one of an XRPD pattern substantially the same as FIG. 4; and/or an XRPD pattern substantially the same as FIG. 5; and/or a DSC profile substantially the same as FIG. 6; and/or a TGA profile substantially the same as FIG. 7. In some embodiments, the aforesaid HCl crystalline salt Form A is characterized by an XRPD pattern comprising four or more peaks, in terms of 2-theta degrees, chosen from peaks at about 9.7±0.2, 14.9±0.2, 17.6±0.2, 19.0±0.2, 19.6±0.2, 21.6±0.2, 22.3±0.2, and 29.3±0.3.

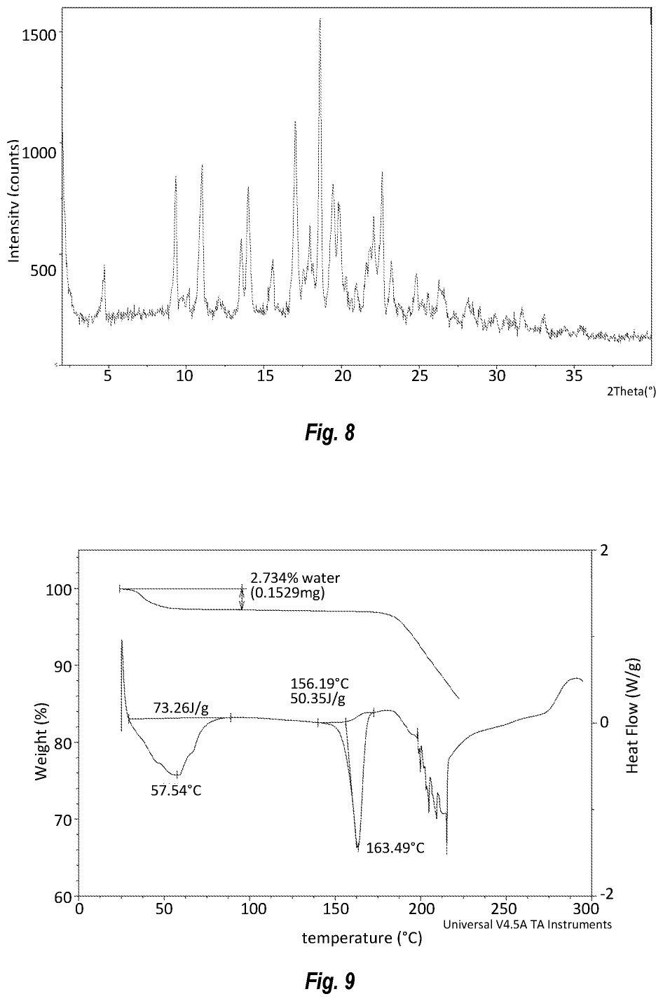

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate birefringent crystalline salt Form A. In some embodiments, the aforesaid oxalate birefringent crystalline salt Form A is characterized by at least one of an XRPD pattern substantially the same as FIG. 8; and/or a DSC/TGA profile substantially the same as FIG. 9.

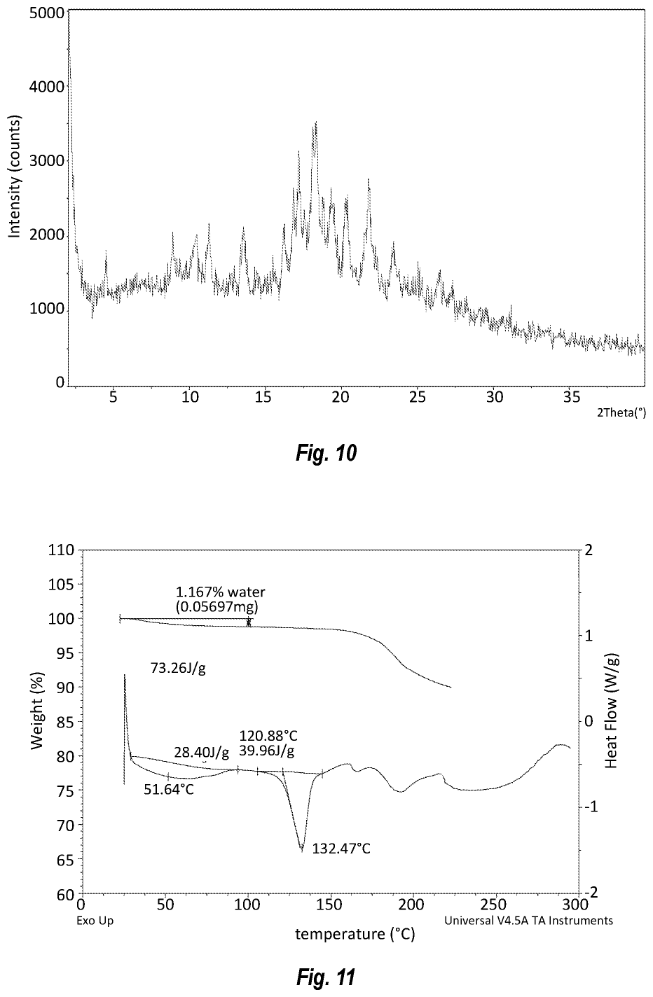

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate birefringent crystalline salt. In some embodiments, the aforesaid maleate birefringent crystalline salt is characterized by at least one of an XRPD pattern substantially the same as FIG. 10; and/or a DSC/TGA profile substantially the same as FIG. 11.

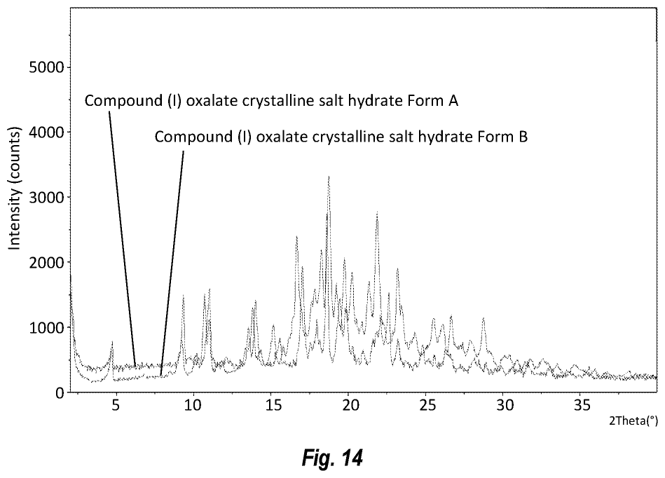

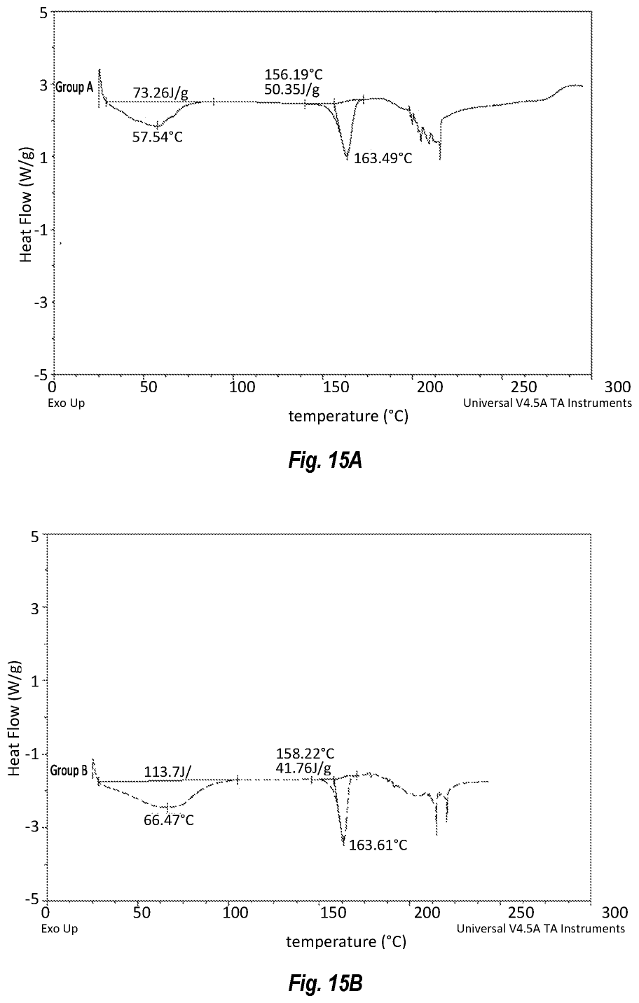

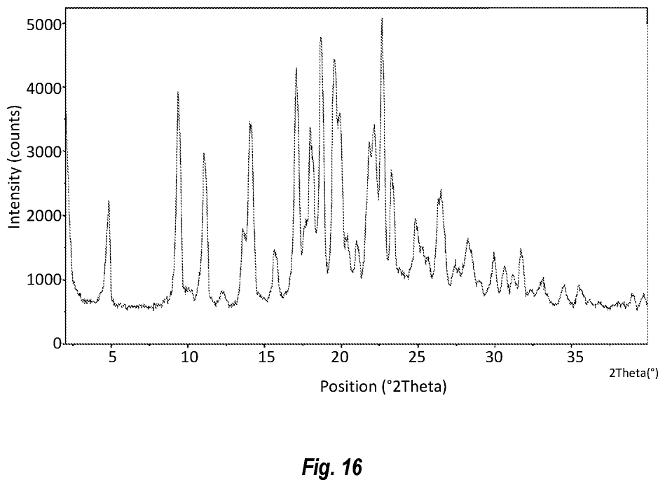

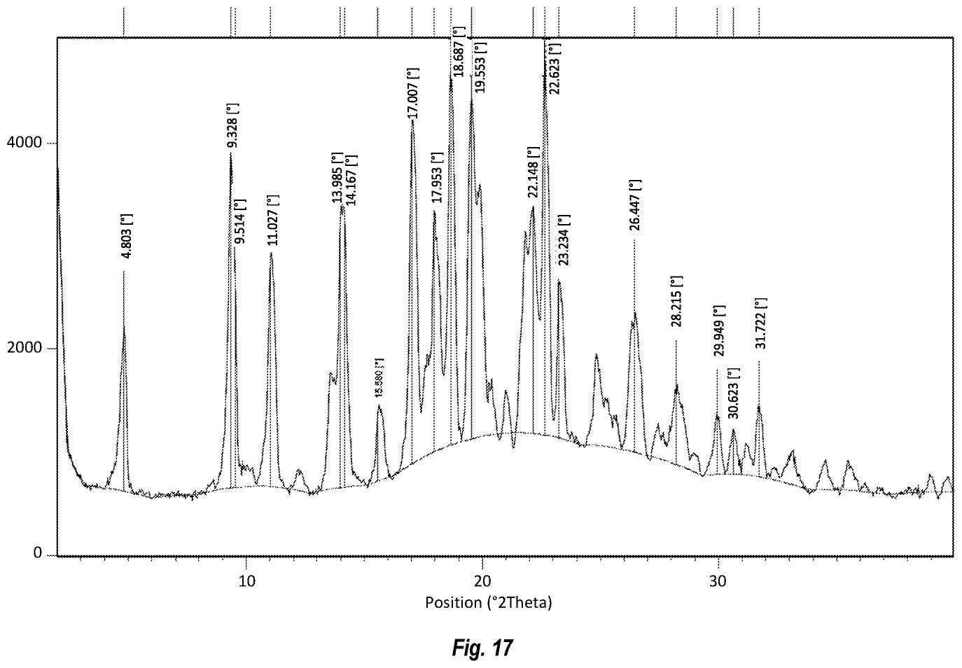

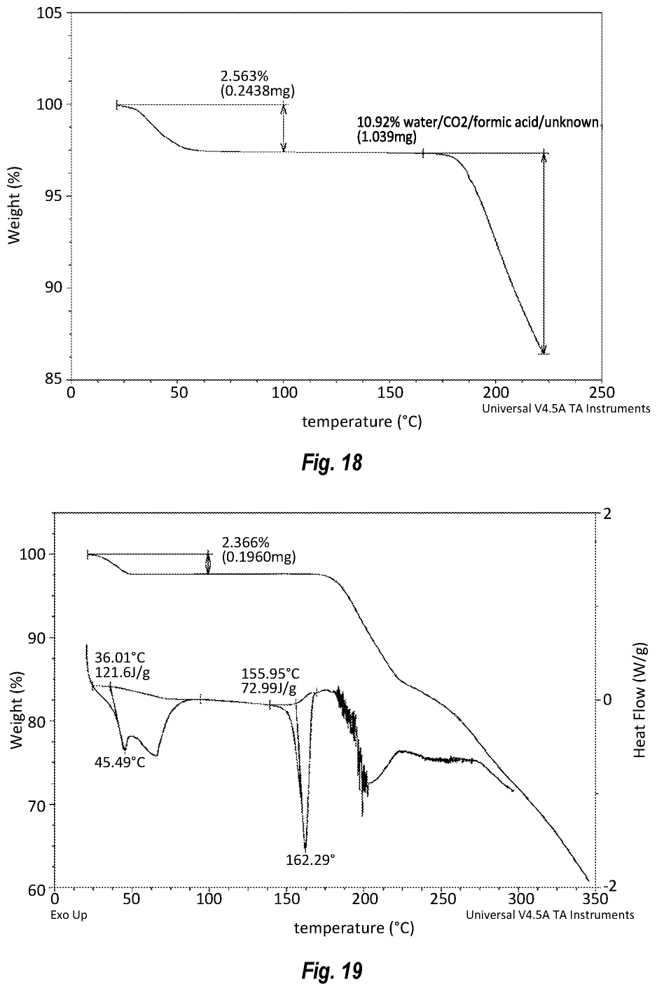

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form A. In some embodiments, the aforesaid oxalate crystalline salt hydrate Form A is characterized by at least one of an XRPD pattern substantially the same as FIG. 14; and/or a DSC profile substantially the same as FIG. 15A. In some embodiments, the aforesaid oxalate crystalline salt hydrate Form A is characterized by at least one of an XRPD pattern substantially the same as FIG. 16; and/or an XRPD pattern substantially the same as FIG. 17; and/or a TGA profile substantially the same as FIG. 18; and/or a DCS/TGA profile substantially the same as FIG. 19. In some embodiments, the aforesaid oxalate crystalline salt hydrate Form A is characterized by an XRPD pattern comprising four or more peaks, in terms of 2-theta degrees, chosen from peaks at about 4.8±0.2, 9.3±0.2, 14.0±0.2, 14.2±0.2, 17.0±0.2, 18.7±0.2, 19.6±0.2, and 22.6±0.2.

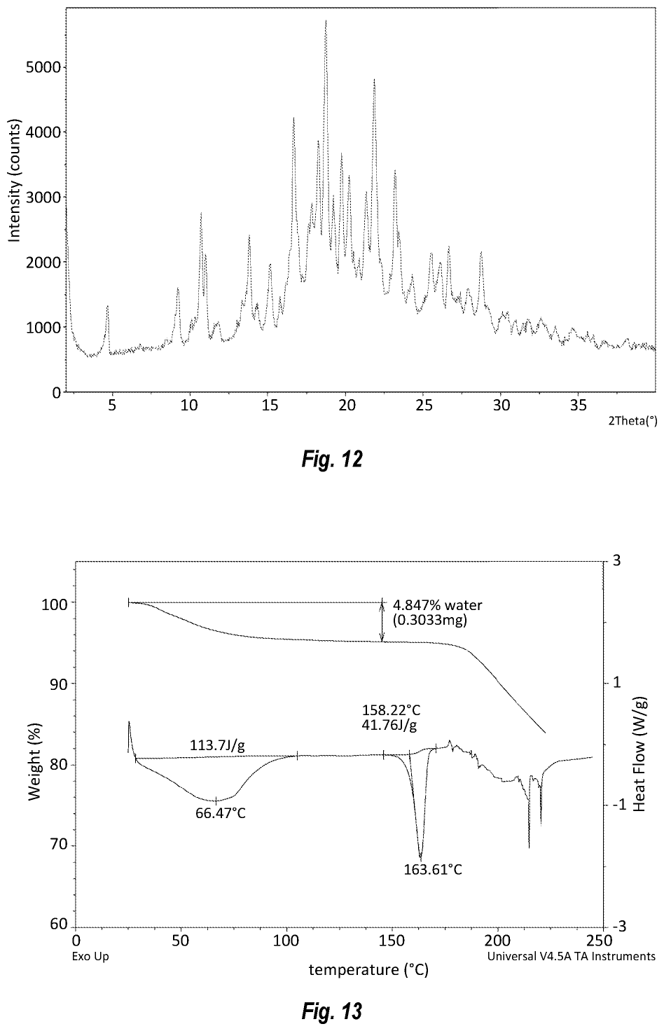

In one embodiment, the present disclosure comprises 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form B. In some embodiments, the aforesaid oxalate crystalline salt hydrate Form B is characterized by at least one of an XRPD pattern substantially the same as FIG. 12; and/or a DSC/TGA profile substantially the same as FIG. 13; and/or a DSC profile substantially the same as FIG. 15B.

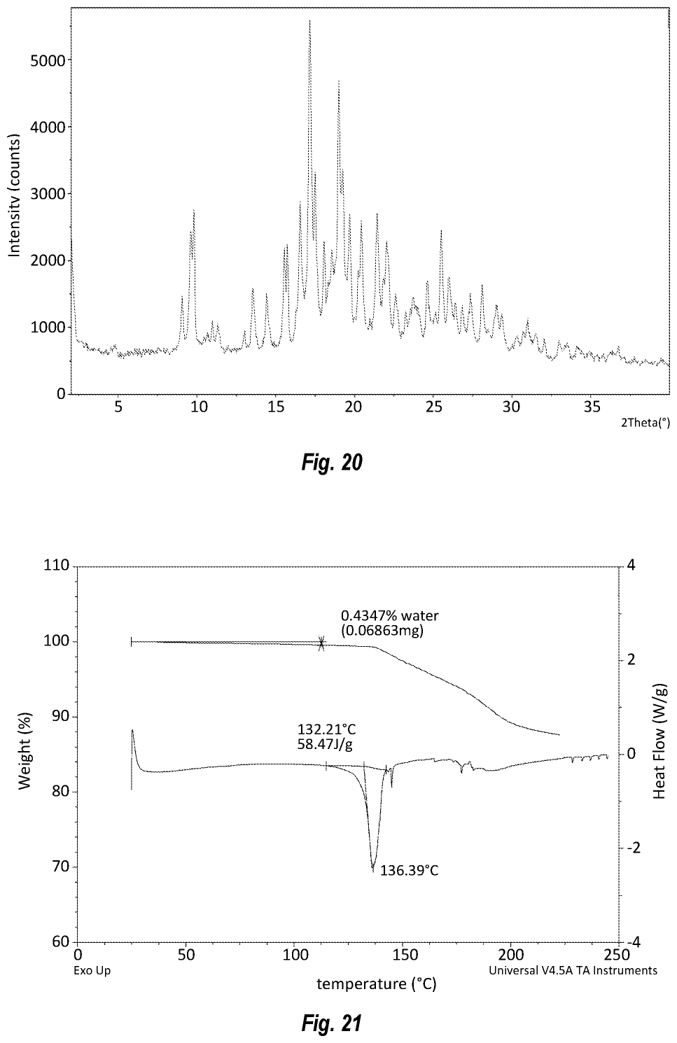

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form B·MeCN solvate. In some embodiments, the aforesaid maleate crystalline salt Form B·MeCN solvate is characterized by at least one of an XRPD pattern substantially the same as FIG. 20; and/or a DSC/TGA profile substantially the same as FIG. 21.

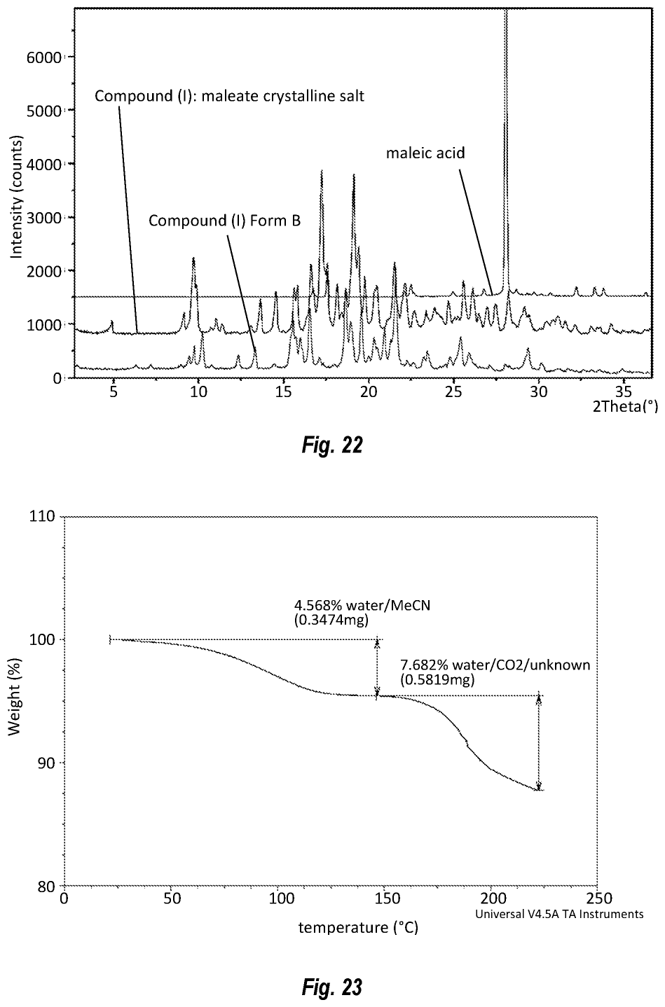

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form A. In some embodiments, the aforesaid maleate crystalline salt Form A is characterized by at least one of an XRPD pattern substantially the same as FIG. 22; and/or a TGA profile substantially the same as FIG. 23. In some embodiments, the aforesaid maleate crystalline salt Form A is characterized by an XRPD pattern comprising four or more peaks, in terms of 2-theta degrees, chosen from peaks at about 9.7±0.2, 14.9±0.2, 17.6±0.2, 19.0±0.2, 19.6±0.2, 21.6±0.2, 22.3±0.2, and 22.8±0.2.

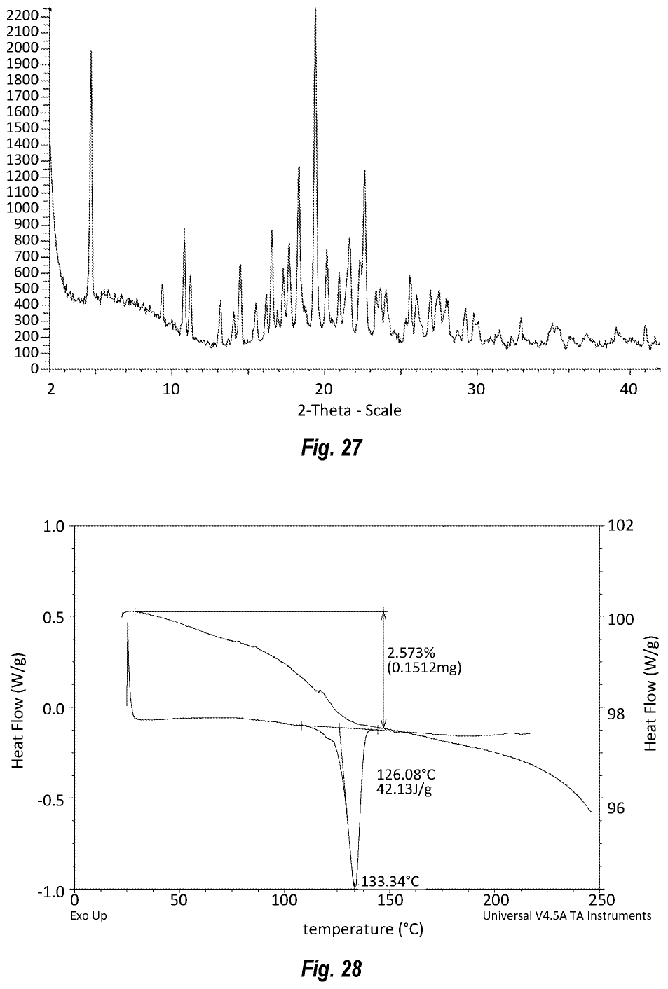

In one embodiment, the present disclosure comprises a substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, which is a methyl paraben co-crystal. In some embodiments, the aforesaid methyl paraben co-crystal of the disclosure is characterized by at least one of an XRPD pattern substantially the same as FIG. 27; and/or a DCS/TGA profile substantially the same as FIG. 28. In some embodiments, the aforesaid methyl paraben co-crystal is characterized by an XRPD pattern comprising four or more peaks, in terms of 2-theta degrees, chosen from peaks at about 4.6±0.2, 10.8±0.2, 16.6±0.2, 18.3±0.2, 19.3±0.2, 20.2±0.2, 21.6±0.2 and 22.5±0.2. In other embodiments, the substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile is chosen from an HCl salt, an oxalate salt, a maleate salt, or a methyl paraben co-crystal, that is at least 50% crystalline, such as at least 60%, at least 70%, at least 80%, at least 90%, at least 95%, at least 96%, at least 97%, at least 98%, at least 99%, or 100% crystalline.

Further embodiments disclosed herein involve pharmaceutical compositions comprising at least one substantially crystalline form of any of the alternative embodiments herein, and a pharmaceutically acceptable excipient; optionally, the pharmaceutical composition is formulated for modified or extended release of a salt form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, to a subject; optionally, for treatment of a disease in a subject that is mediated by the BTK kinase.

### Pharmaceutical Compositions

The crystalline forms described herein are useful as active pharmaceutical ingredients (APIs), as well as materials for preparing pharmaceutical compositions that incorporate one or more pharmaceutically acceptable excipients and are suitable for administration to human subjects. In some embodiments, these pharmaceutical compositions will be a pharmaceutical product, such as, e.g., a solid oral dosage form, such as tablets and/or capsules.

In some embodiments, the present disclosure provides a pharmaceutical composition comprising at least one crystalline form of Compound (I) selected from an HCl salt, an oxalate salt, a maleate salt, or a methyl paraben co-crystal. In some embodiments, the present disclosure provides a pharmaceutical composition comprising at least one crystalline form of Compound (I) as described herein and at least one additional pharmaceutically acceptable excipient. Each excipient must be “pharmaceutically acceptable” in the sense of being compatible with the subject composition and its components not injurious to the patient. Except insofar as any conventional pharmaceutically acceptable excipient is incompatible with Compound (I), such as by producing any undesirable biological effect or otherwise interacting in a deleterious manner with any other component(s) of the pharmaceutically acceptable composition, its use is contemplated to be within the scope of this disclosure.

In some embodiments, the present disclosure provides a pharmaceutical composition comprising at least one crystalline form of Compound (I) selected from an HCl salt, an oxalate salt, a maleate salt, or a methyl paraben co-crystal, formulated with one or more excipients for use in providing extended release or modified release dosage administration to a subject.

Some non-limiting examples of materials which may serve as pharmaceutically acceptable excipients include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non toxic compatible substances employed in pharmaceutical formulations. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of which are incorporated by reference herein, also disclose additional non-limiting examples of pharmaceutically acceptable excipients, as well as known techniques for preparing and using the same.

Pharmaceutical compositions disclosed herein may be administered orally, parenterally, by inhalation spray, topically, rectally, nasally, buccally, vaginally, or via an implanted reservoir. The term “parenteral,” as used herein includes subcutaneous, intravenous, intramuscular, intra articular, intra synovial, intrasternal, intrathecal, intrahepatic, intralesional, and intracranial injection or infusion techniques. In some embodiments, the compositions of the disclosure are administered orally, intraperitoneally, or intravenously. Sterile injectable forms of the pharmaceutical compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3 butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono or di glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween, Spans, and other emulsifying agents or bioavailability enhancers which are commonly used in the manufacture of pharmaceutically acceptable solid, liquid, or other dosage forms may also be used for the purposes of formulation.

Pharmaceutical compositions disclosed herein may also be orally administered in any orally acceptable dosage form including, but not limited to, capsules, tablets, aqueous suspensions, or solutions. When aqueous suspensions are required for oral use, the active ingredient is typically combined with emulsifying and suspending agents. If desired, certain sweetening, flavoring, or coloring agents may also be added.

In some embodiments, the pharmaceutical compositions comprising the crystalline salt forms disclosed herein may be used as inhibitors of the Bruton's tyrosine kinase (BTK) and in treating a disease mediated by the BTK in a mammal in need thereof. Such diseases and methods of treatment with the crystals disclosed herein may be selected from acute necrotizing hemorrhagic leukoencephalitis, acute disseminated encephalomyelitis, autoimmune inner ear disease (AIED), autoimmune retinopathy, axonal & neuronal neuropathies, chronic inflammatory demyelinating polyneuropathy (CIDP), demyelinating neuropathies, Devic's disease (neuromyelitis optica), experimental allergic encephalomyelitis, giant cell arteritis (temporal arteritis), Guillain-Barre syndrome, Lambert-Eaton syndrome, chronic Meniere's disease, myasthenia gravis, neuromyotonia, opsoclonus-myoclonus syndrome, optic neuritis, paraneoplastic cerebellar degeneration, peripheral neuropathy, perivenous encephalomyelitis, restless legs syndrome, stiff person syndrome, sympathetic ophthalmia, Takayasu's arteritis, temporal arteritis/Giant cell arteritis, transverse myelitis, multiple sclerosis, dysautonomia, age-related macular degeneration (wet and dry), corneal transplantation, encephalitis, meningitis, vasculitis, or systemic lupus erythematosus (SLE); rheumatoid arthritis, psoriatic arthritis, atopic dermatitis, lupus, uveitis, myasthenia gravis, warm autoimmune hemolytic anemia, immune thrombocytopenia (ITP), Wegener's granulomatosis, Sjogren's disease, Sjogren's dry eye, non-Sjogren's dry eye disease, psoriasis, pemphigus, urticaria (such as chronic spontaneous urticaria), asthma, diseases related to IgG4 regulation, diffuse large B cell lymphoma, follicular lymphoma, chronic lymphocytic lymphoma, chronic lymphocytic leukemia, B-cell prolymphocytic leukemia, small lymphocytic lymphoma (SLL), multiple myeloma, B-cell non-Hodgkin lymphoma, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, and lymphomatoid granulomatosis.

### EXAMPLES

As used in the following examples or elsewhere herein, the following Table of Abbreviations may be useful.

Abbreviations

| | |
|---|---|
| AcOEt | ethyl acetate |
| Al | aluminum |
| CI | counter-ion |
| DBU | 1,8-diazabicyclo[5.4.0]undec-7-ene |
| DCM | dichloromethane |
| DIEA | diethylamine |
| DIPEA | diisopropylethylamine |
| DMC | dimethyl carbonate |
| DMF/DMA | dimethylformamide/dimethylacetamide |
| DSC | differential scanning calorimetry |
| EtOAc | ethyl acetate |
| EtOH | ethanol |
| Eq. or equiv. | equivalent |
| EV | evaporation |
| FEV | fast evaporation |
| FT | Fourier transform |
| ¹HNMR | proton nuclear magnetic resonance |
| HPLC | high-performance liquid chromatography |
| hr | hour(s) |
| IPE | isopropyl ether |
| iPrOAc | isopropylacetate |
| MeCN | acetonitrile |
| MeOH | methanol |
| MIBK | 4-methyl-2-pentanone |
| min | minute(s) |
| mM | millimole |
| MTBE | methyl tert-butyl ether |
| PLM | polarized light microscopy |
| Pt | platinum |
| RC | rapid cooling |
| RT | room temperature (or ambient temperature) |
| T3P | propylphosphonic anhydride |
| TA | Texas Analytics |
| TC | temperature cycling |
| TG | thermogravimetry |
| TGA | thermogravimetric analysis |
| vol. | volume |
| Wt. | weight |
| XRPD | x-ray powder diffraction |

### Instrumentation

The following instrumentation and procedures were used to collect the data set forth in the Examples herein. One skilled in the field can appreciate that alternate instrumentation and procedures as available and known to the skilled artisan optionally may be used to collect characterization data such as NMR, PLM, XRPD, TGA, and DSC/TGA data.

### Option A Instrumentation

XRPD. XRPD data was collected with a PANalytical X′Pert Pro diffractometer using Ni-filtered Cu Ka (45 kV/40 mA) radiation and a step size of 0.02° 2θ and X′celerator™ RTMS (Real Time Multi-Strip) detector. Configuration on the incidental beam side: fixed divergence slit (0.25°), 0.04 rad Soller slits, anti-scatter slit (0.25°), and 10 mm beam mask. Configuration on the diffracted beam side: fixed divergence slit (0.25°) and 0.04 rad Soller slit. Samples were mounted flat on zero-background Si wafers.

DSC. Data was collected with a TA Instruments Q100 or Q2000 differential scanning calorimeter equipped with an autosampler and a refrigerated cooling system under a 40 mL/min N₂ purge. DSC thermograms of the samples were obtained at 10° C./min in crimped Al pans.

TGA. Data was collected with a TA Instruments Q50 thermogravimetric analyzer under a 40 mL/min N₂ purge in Pt or Al pans. TGA thermograms of the samples were obtained at 10° C./min.

### Option B Instrumentation

XRPD. XRPD data alternatively was collected with a Bruker AXS C2 General Area Detector Diffraction System (GADDS) diffractometer using Cu Kα radiation (40 kV, 40 mA), an automated XYZ stage, a laser video microscope for auto-sample positioning and a Våntec-500 2-dimensional area detector. X-ray optics consists of a single Göbel multilayer mirror coupled with a pinhole collimator of 0.3 mm. The beam divergence, i.e., the effective size of the X-ray beam on the sample, was approximately 4 mm. A θ-θ continuous scan mode was employed with a sample—detector distance of 20 cm which gives an effective 2θ range of 1.5°-32.5°. Typically, the sample was exposed to the X-ray beam for 120 seconds. The software used for data collection and analysis was Bruker GADDS for Win7/XP and Diffrac Plus EVA respectively. Samples run under ambient conditions were prepared as flat plate specimens using powder as received without grinding. Samples were prepared and analysed on a glass slide, by lightly pressed the powder to obtain a flat surface for analysis.

DSC. Data alternatively was collected on a TA Instruments Discovery DSC equipped with a 50-position auto-sampler. Typically, 0.5-3 mg of each sample, in a pin-holed aluminum pan, was heated at 10° C./min from 25° C. to around 230° C. A purge of dry nitrogen at 50 ml/min was maintained over the sample. The instrument control software was TRIOS and the data was analyzed using TRIOS or Universal Analysis.

TGA. TGA data was collected on a TA Instruments Discovery TGA, equipped with a 25-position auto-sampler. Typically, 5-10 mg of each sample was loaded onto a pre-tared aluminum DSC pan and heated at 10° C./min from ambient temperature to 350° C. A nitrogen purge at 25 ml/min was maintained over the sample. The instrument control software was TRIOS and the data were analyzed using TRIOS or Universal Analysis.

PLM. Samples were analyzed on a Leica LM/DM polarized light microscope with a digital video camera for image capture. A small amount of each sample was placed on a glass slide, with or without immersion oil, and covered with a glass slip. The sample was viewed with appropriate magnification and partially polarized light, coupled to a λ false-color filter. Images were captured using Studio Capture or Image ProPlus software.

NMR. ¹H NMR spectra were collected on a Bruker 400 MHz instrument equipped with an auto-sampler and controlled by a DRX400 console. Samples typically were prepared in DMSO-d6 solvent. Automated experiments were acquired using ICON-NMR configuration within Topspin software, using standard Bruker-loaded experiments (1H). Off-line analysis was performed using ACD Spectrus Processo.

### Starting Material—Exemplary Synthesis of Compound (1) Form B

Form B of Compound (I) disclosed in US 2021/0221818 (corresponding to WO2021/150723) was used as the starting material in each of the procedures described in Examples 1 through 9 below and as a comparative sample in Examples 10 to 14. Form B of Compound (I) can be prepared according to the procedures set forth in Examples 2 through 4 of US 2021/0221818 (corresponding to WO2021/150723), optionally, according to Example 4 therein, which is incorporated herein by reference.

The following preparations of Compound (I) Form B disclosed in US 2021/0221818 (corresponding to WO2021/150723) are provided to enable those skilled in the art to prepare the BTK inhibitor compound. The synthetic route should not be considered as limiting the scope of the disclosure, but merely as being illustrative and representative thereof.

In one alternative, 96 mg of amorphous (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-5 enenitrile was dissolved in 0.3 mL ethyl acetate. The obtained solution was seeded with NaCl and stirred at room temperature. After overnight stirring, a cloudy solution was obtained and sonicated for 5 minutes. After an additional two days of stirring, a suspension was obtained and filtered (centrifugal unit filter, PTFE, 0.22 μm) to obtain crystalline Form B disclosed in US 2021/0221818 (corresponding to WO2021/150723).

In another alternative, 430 g of Form C of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (Compound (I)) was combined with ethanol (4.1 L) at approximately 15° C. to form a slurry. Form B disclosed in US 2021/0221818 (corresponding to WO2021/150723) seed crystal was then added (to approximately 5 wt. %), and the slurry was stirred for approximately two days. The slurry was filtered and dried under vacuum with heat to obtain approximately 300 g of crystalline Form B of Compound (I) disclosed in US 2021/0221818 (corresponding to WO2021/150723) (74% yield).

Form C can also be prepared according to the procedures set forth in Examples 5 through 8 of US 2021/0221818 (corresponding to WO2021/150723), for example, as described in Example 5 therein, which is incorporated herein by reference. 100 mg of amorphous (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile was combined with acetonitrile (0.5 mL). The solution was seeded with crystalline Form B of Compound (I) disclosed in US 2021/0221818 (corresponding to WO2021/150723) and stirred at RT for 48 hrs. At about 48 hrs., a thick white free-flowing slurry was obtained and determined to be crystalline Form C (estimated yield >50%).

### Example 1—Solubility and Solvent Assessment

Solubility of the Form B crystal of Compound (I) disclosed in US 2021/0221818 (corresponding to WO2021/150723) was assessed in a diverse variety of solvents to facilitate the selection of solvent systems and corresponding dosing strategies for the subsequent screening experiments of Example 2. The solubility was visually estimated in 12 common solvents at RT. Form B crystal disclosed in US 2021/0221818 (corresponding to WO2021/150723) (˜20 mg) was weighed into each of 12 vials, followed by addition of 200 μL of solvent at RT and observed for 15 min. If the solid did not dissolve in 200 μL at RT, a second aliquot of 800 μL of solvent was added at RT and checked visually for dissolution. The results are shown in Table 1.

TABLE 1
Visual Solubility of Form B Crystal of Compound (I)

| # | Solvent | Solubility at RT [mg/mL] |
|---|---|---|
| 1 | Acetone | >100 |
| 2 | Dichloromethane (DCM) | >100 |
| 3 | Tetrahydrofuran (THF) | >100 |
| 4 | Acetonitrile (MeCN) | 20-100 |
| 5 | Ethyl acetate (EtOAc) | 20-100 |
| 6 | Dimethyl carbonate (DMC) | 20-100 |
| 7 | Cyclohexanone | 20-100 |
| 8 | 4-Methyl-2-pentanone (MIBK) | 20-100 |
| 9 | Toluene | <20 |
| 10 | t-Butyl Methyl Ether (MTBE) | <20 |
| 11 | Isopropyl ether (IPE) | <20 |
| 12 | Cyclohexane | <20 |

### Example 2—Compound (I): HCl Birefringent Crystalline Salt

### 2.1 HCl Crystal Screen.

Twelve (12) experiments were conducted using the Form B crystal of Compound (I) disclosed in US 2021/0221818 (corresponding to WO2021/150723) with a focus on the HCl salt. The experiments were designed to generate data as to whether, and under what conditions, the HCl crystallization salt may be obtained in various solvent systems and experimental conditions and the nature of the product produced. This experiment was conducted using solutions/gums/suspensions that were generated from the solubility experiments of Example 1 as starting material. One equivalent of 4M HCl in 1,4-dioxane was dosed to the 12 samples produced during said assessment (Example 1) in 12 aprotic solvents (MeCN, acetone, DCM, MIBK, EtOAc, THF, IPE, toluene, cyclohexane, DMC, MTBE, cyclohexanone), and a five-step crystallization procedure was then utilized. The experiments proceeded as follows:
1. One equivalent of HCl in 1,4-dioxane was added to the solutions/gums/suspensions obtained from the 12 solubility experiments of Example 1;
2. The solutions/gums/suspensions were stirred while the temperature was cycled between 25° C. and 5° C. for two days (TC1 crystallization);
3. The solvents were fast evaporated under reduced pressure via GeneVac and 200 μL of the solvents was redispensed (FEV);
4. The solutions/gums/suspensions were stirred while the temperature was cycled between 25° C. and 5° C. for five days (TC2 crystallization);
5. The solutions/gums/gels were stirred at 25° C. for an hour, cooled to 4° C. and held at 4° C. for two days (RC);
6. The solvent was evaporated at RT in a N₂ bleed chamber for five days (EV).

PLM and XRPD analyses were conducted on each of the 12 samples in situ at each mode of crystallization (TC1, FEV, TC2, RC, and EV). The results are reported below in Table 2. Based on the PLM and XRPD results, birefringent (i.e. “double refraction”) crystals were obtained in two instances only: (1) following TC2 (step 4) using MeCN as solvent, and (2) following EV (step 5) using DMC as solvent. The remaining experiments produced either the Form B crystal disclosed in US 2021/0221818 (corresponding to WO2021/150723) or the amorphous form of Compound (I). The XRPD patterns of the two birefringent samples were obtained and are reported in FIG. 1. As indicated by the XRPD patterns, the two samples had substantially the same form. Thermal analysis also suggested they comprised a hydrated form.

TABLE 2
Results from Crystal Screening Experiments with HCl

| Sample | | | |
|---|---|---|---|
| No. | Solvent | Mode of Crystallization | Result |
| 1 | MeCN | TCI, FEV, TC2 | Birefringent crystal |
| 2 | Acetone | TCI, FEV, TC2, RC, EV | amorphous |
| 3 | DCM | TCI, FEV, TC2, RC, EV | amorphous |
| 4 | MIBK | TCI | amorphous |
| 5 | EtOAc | TCI | amorphous |
| 6 | THF | TCI, FEV, TC2, RC, EV | amorphous |
| 7 | IPE | TCI | Form B |
| 8 | Toluene | TCI | amorphous |
| 9 | Cyclohexane | TCI | amorphous |
| 10 | DMC | TCI, FEV, TC2, RC, EV | Birefringent crystal |
| 11 | MTBE | TC1 | Form B |
| 12 | Cyclohexanone | TCI, FEV, TC2, RC, EV | amorphous |

### 2.2. Scale Up and Characterization of Compound (I): HCl Crystalline Salt Form A.

The birefringent crystalline HCl salt product obtained from Example 2.1 was scaled up to 100 mg and 1.4 g and further characterized. For the 100 mg-scale, about 100.6 mg of Compound (I) Form B crystal disclosed in US 2021/0221818 (corresponding to WO2021/150723) was mixed in MeCN (1 mL) at RT to form a slurry. To the slurry was added 4M HCl solution in 1,4-dioxane (1 eq). The resulting solution was seeded with the birefringent HCl crystalline salt product (1 mg) obtained from Example 2.1; seed persisted. The slurry was stirred at RT for 2 days. The solids were filtered and air-dried for 2 hr to yield 78 mg of Compound (I) HCl crystalline salt Form A.

### 2.3. Preparation and Characterization of Compound (I): HCl Crystalline Salt Hydrate.

About 1.6 g of Compound (I) Form B disclosed in US 2021/0221818 (corresponding to WO2021/150723) was mixed in MeCN (16 mL) at RT. To this slurry was added 4M HCl in 1,4-dioxane (1 eq). The solution was seeded with Compound (I) HCl crystalline salt Form A (70 mg); seed persisted. The slurry was stirred at RT for 3 days. The solids were filtered and air-dried for 16 hr to yield 1.4 g of product comprising Compound (I) mono-HCl crystalline salt as a hydrate (1.9 eq.) (90% yield). The XRPD of the sample is shown in FIG. 2 confirming the crystalline product. The results of DSC and TGA thermograms are shown in FIG. 3; DSC showed a broad endotherm between 25-140° C. and an endotherm with onset at 160.8° C. TGA showed 4.6% weight loss of water (1.9 eq.) between 25-140° C. HPLC purity was 99.4% of the E-isomer.

### Example 3—Preparation and Characterization of Compound (I) HCl Crystalline Salt Form A

Approximately 10 g of Compound (I) Form B freebase disclosed in US 2021/0221818 (corresponding to WO2021/150723) was stirred in acetonitrile (approximately 100 mL) leading to a suspension. A saturated HCl solution in isopropanol (approximately 3 mL) was added. After approximately 3 min, a suspension formed. The slurry was stirred for approximately 3 days, then filtered and washed with acetonitrile. The resultant crystals were dried under vacuum at 30° C. to provide 7.8 g of the HCl crystalline salt Form A of Compound (I) as an anhydrous white solid with 99.7% purity of the E isomer of Compound (I) and 99.8% purity of the combined E and Z isomers.

### 3.1. XRPD for Example 3 (Compound (I) HCl Crystalline Salt Form A).

XRPD diffractograms of Compound (I): HCl crystalline salt Form A were obtained with the instrumentation and procedures set forth previously in the Instrumentation Section (Option A). The XRPD patterns set forth in FIGS. 4 and 5 were obtained in repeated experiments with FIG. 5 displaying the peak numbers. Peaks identified in FIG. 5 include those listed in Tables 3A and 3B, below; optionally, the peaks reported in Table 3B may be considered as representative.

TABLE 3A
XRPD Data for Example 3 (Compound
(I) HCl Crystalline Salt Form A)

| Pos. | Height | d-spacing | Rel. Int. |
|---|---|---|---|
| [°2Th.] | [cts] | [Å] | [%] |
| 9.7465 | 1562.11 | 9.07496 | 17.54 |
| 10.4788 | 1105.89 | 8.44242 | 12.41 |
| 13.6342 | 873.57 | 6.49482 | 9.81 |
| 14.4184 | 1073.08 | 6.14329 | 12.05 |
| 14.8560 | 1454.21 | 5.96329 | 16.32 |
| 15.7268 | 891.13 | 5.63501 | 10.00 |
| 17.2514 | 1142.97 | 5.14030 | 12.83 |
| 17.5655 | 2512.19 | 5.04909 | 28.20 |
| 17.9223 | 820.16 | 4.94938 | 9.21 |
| 18.9759 | 3010.34 | 4.67689 | 33.79 |
| 19.5772 | 8908.41 | 4.53456 | 100.00 |
| 20.9373 | 890.00 | 4.24298 | 9.99 |
| 21.5990 | 6909.03 | 4.11446 | 77.56 |
| 22.3540 | 3830.81 | 3.97716 | 43.00 |
| 22.7954 | 1963.33 | 3.90114 | 22.04 |
| 25.3508 | 1192.24 | 3.51340 | 13.38 |
| 26.4252 | 939.75 | 3.37294 | 10.55 |
| 27.4237 | 505.54 | 3.25236 | 5.67 |
| 29.1370 | 936.56 | 3.06490 | 10.51 |
| 29.3288 | 1526.16 | 3.04529 | 17.13 |

TABLE 3B
XRPD Data for Example 3 (Compound
(I) HCl Crystalline Salt Form A)

| Pos. | Height | d-spacing | Rel. Int. |
|---|---|---|---|
| [°2Th.] | [cts] | [Å] | [%] |
| 9.7465 | 1562.11 | 9.07496 | 17.54 |
| 14.8560 | 1454.21 | 5.96329 | 16.32 |
| 17.5655 | 2512.19 | 5.04909 | 28.20 |
| 18.9759 | 3010.34 | 4.67689 | 33.79 |
| 19.5772 | 8908.41 | 4.53456 | 100.00 |
| 21.5990 | 6909.03 | 4.11446 | 77.56 |
| 22.3540 | 3830.81 | 3.97716 | 43.00 |
| 22.7954 | 1963.33 | 3.90114 | 22.04 |
| 29.3288 | 1526.16 | 3.04529 | 17.13 |

### 3.2. TGA Profile for Example 3 (Compound (I) HCl Crystalline Salt Form A).

TGA thermograms of Example 3 (Compound (I) HCl crystalline salt Form A) were obtained with the Instrumentation and procedures described above in the Instrumentation Section (Option A). Results are shown in FIG. 6.

### 3.3. DSC/TGA Thermogram of Compound (I): HCl Crystalline Salt Form A

DSC was conducted on samples of Example 3 with the Instrumentation and procedures set forth above in the Instrumentation Section (Option A). Results are shown in FIG. 7; the temperatures of exothermic and endothermic transitions recorded via DSC analysis are shown in FIG. 7 as onset values.

### Example 4—Initial Counterion Study: Oxalate and Maleate Crystalline Salts

Forty-eight (48) crystallization experiments were performed with Compound (I), crystalline Form B disclosed in US 2021/0221818 (corresponding to WO2021/150723), utilizing eight (8) counterions and six (6) solvents. The salt study experiments were conducted using substantially pure E-isomer of Compound (I) crystalline Form B. Compound (I) Form B has two ionizable basic sites with calculated pKₐ values of 5.3 and 3.6. A total of 8 counterions exhibiting suitable pKₐ values were selected and dosed as anhydrous solids (1 eq.) to study the salt formation. Table 4 summarizes the counterions and equivalents used.

TABLE 4
Counterions Used in the 48 Salt Study Experiments

| No. | Counterion | Eq | Dosing Method | pKa⁶ |
|---|---|---|---|---|
| 1 | Pamoic acid | 1 | Anhydrous Solid | 2.5, 3.1 |
| 2 | Citric acid | 1 | Anhydrous Solid | 3.1, 4.8, 6.4 |
| 3 | (+)-L-Tartaric acid | 1 | Anhydrous Solid | 3.0, 4.4 |
| 4 | L-Glutamic acid | 1 | Anhydrous Solid | 2.2, 4.3, 9.7(B) |
| 5 | Malonic acid | 1 | Anhydrous Solid | 2.8, 5.7 |
| 6 | Fumaric acid | 1 | Anhydrous Solid | 3.0, 4.4 |
| 7 | Oxalic acid | 1 | Anhydrous Solid | 1.3, 4.3 |
| 8 | Maleic acid | 1 | Anhydrous Solid | 1.9, 6.2 |

Six aprotic solvents (MeCN, toluene, MIBK, EtOAc, DMC, acetone) were selected for the salt study experiments considering the solubility of the Compound (I) Form B crystal disclosed in US 2021/0221818 (corresponding to WO2021/150723) (Example 1, Table 1), polarity, and chemical diversity. The five-step procedure set forth below was used, and the samples were examined for birefringence by PLM in situ at each step. If a sample was birefringent, the crystals were isolated, analyzed and grouped by XRPD patterns. All XRPD patterns were compared with XRPD patterns for the Compound (I) Form B crystal disclosed in US 2021/0221818 (corresponding to WO2021/150723) and the appropriate counterions for identification:
1 One eq. of the counterion as anhydrous solid was added into a 2-mL HPLC vial containing Form B crystal (˜20 mg);
2. 200-300 μL of solvent (selected from MeCN, toluene, MIBK, EtOAc, DMC, and acetone), was dispensed into the 2-mL HPLC vials each containing a mixture of Form B and counterion as per step 1;
3. The solutions/suspensions/gums were stirred while the temperature was cycled between 25° C. and 5° C. for fifteen days (TC);
4. The solutions/gums obtained from step 3 were stirred at 25° C. for an hour, cooled to 4° C. and held at 4° C. for three days (RC); and
5. The solvent was evaporated at RT in a N₂ bleed chamber for up to 10 days (EV).

Table 5 shows the outcome from this salt study. Of the 48 experiments, only two produced a new crystalline form of Compound (I), namely, the vials containing maleic acid and oxalic acid counterions with acetonitrile as solvent. Thermal analyses revealed the hits were hydrated forms. Other birefringent products were observed and isolated in 31 experiments; however, they were confirmed via XRPD as the Compound (I) Form B crystal disclosed in US 2021/0221818 (corresponding to WO2021/150723), a crystalline counterion, or a mixture of Compound (I) Form B and counterion (designated in Table 4 as “mixture”).

The remaining experiments yielded amorphous products as shown in Table 5.

TABLE 5
Results of 48 Salt Experiments

| | Solvent | | | | | |
|---|---|---|---|---|---|---|
| Counterion | MeCN | Toluene | MIBK | EtOAc | DMMC | Acetone |
| Pamoic | counterion | mixture | mixture | counterion | counterion | counterion |
| acid | | | | | | |
| Citric | counterion | mixture | mixture | counterion | counterion | amorphous |
| acid | | | | | | |
| {+)-L- | counterion | Form B | mixture | counterion | counterion | amorphous |
| Tartaric | | | | | | |
| acid | | | | | | |
| L- | mixture | mixture | mixture | mixture | mixture | mixture |
| Glutamic | | | | | | |
| acid | | | | | | |
| Malonic | amorphous | amorphous | amorphous | amorphous | amorphous | amorphous |
| acid | | | | | | |
| Fumaric | counterion | mixture | mixture | counterion | counterion | amorphous |
| acid | | | | | | |
| Oxalic | birefringent | Form B | amorphous | Form B | amorphous | amorphous |
| acid | crystal | | | | | |
| Maleic | birefringent | mixture | counterion | amorphous | amorphous | amorphous |
| acid | crystal | | | | | |

### 4.1 Oxalate Birefringent Salt Form A

Compound (I): oxalate salt birefringent crystal generated in acetonitrile was a hydrate (2.7% wt. water). The XRPD pattern is shown in FIG. 8, and DSC and TGA thermograms are shown in FIG. 9. The DSC thermogram showed a broad endotherm between 25-90° C. and an endotherm with onset at 156.2° C. HPLC purity was 99.8% of the E-isomer.

### 4.2 Maleate Birefringent Salt

Compound (I): maleate salt birefringent crystal generated in acetonitrile was a poorly crystalline solid as reflected by the XRPD pattern shown in FIG. 10. The DSC and TGA thermograms of the maleate salt are shown in FIG. 11. The DSC thermogram showed a broad endotherm between 25-100° C. and an endotherm with onset at 120.9° C. HPLC purity was 99.8% of the E-isomer.

### Example 5—Compound (I) Oxalate Crystalline Salt Forms A and B

### 5.1 Compound (I) Oxalate Crystalline Salt Hydrate Form A

Scale up and characterization of the oxalate salt from Example 4.1 generated two forms of crystalline salt product, designated herein as Compound (I) oxalate crystalline salt hydrate Form A and Compound (I) oxalate crystalline salt hydrate Form B.

A 100-mg scale up was performed by stirring Compound (I) Form B disclosed in US 2021/0221818 (corresponding to WO2021/150723) (100.4 mg) and oxalic acid solid (1 eq.) in MeCN (1 mL) at RT. The slurry was seeded with the oxalate salt product of Example 4.1 (1 mg); seed persisted. The mixture was stirred at RT for 2 days. An XRPD of an aliquot sample showed a new Compound (I) oxalate crystalline salt Form B which was converted to Form A, after drying in 30° C. for 7 hr. The solids were filtered and air-dried for 4 hr to give Compound (I) oxalate crystalline salt hydrate Form A. Yield was 70% (80 mg).

5.2 Compound (I) Oxalate Crystalline Salt hydrate Form B

Additional preparation and characterization were performed. Form B of Compound (I) disclosed in US 2021/0221818 (corresponding to WO2021/150723) (1.57 g) and oxalic acid solid (1 eq.) were stirred in MeCN (16 mL) at RT, the slurry was seeded with the oxalate salt of Example 4.1 (70 mg); seed persisted. The mixture was stirred at RT for 3 days. An XRPD of an aliquot sample showed Compound (I) oxalate crystalline salt hydrate Form B. The solids were filtered and air-dried for 16 hr to still yield the Compound (I) oxalate crystalline salt Form B (1.52 g; 85% yield), which also was a hydrate (4.8% wt water) and is characterized by the XRPD pattern shown in FIG. 12. DSC and TGA thermograms for Compound (I) oxalate crystalline salt hydrate Form B are shown in FIG. 13. An overlay of the XRPD patterns for Compound (I) oxalate crystalline salt hydrate Forms A and B is shown in FIG. 14. The DSC of Compound (I) oxalate crystalline salt hydrate Forms A and B are shown in FIGS. 15A and 15B, respectively.

The DSC thermogram for Compound (I) oxalate crystalline salt hydrate Form B showed a broad endotherm between 25-110° C. and an endotherm with onset at 158.2° C. HPLC purity was 99.5% of the E-isomer. Ion chromatography data indicated a mono-oxalate salt (theoretical 11.78% wt CI vs experimental 11.42% wt CI). Attempts to make Compound (I) oxalate crystalline salt Form A from the oxalate crystalline salt Form B (prepared as described in Example 5.2) by drying the scaled-up product under vacuum (10-in Hg) at 30° C. with a N₂ bleed for 24 hr yielded a poorly crystalline solid which was a mixture of Example 4.1 (oxalate birefringent salt Form A), Example 5.2 (oxalate crystalline salt hydrate Form B), and amorphous product.

### Example 6—Compound (I) Oxalate Crystalline Salt Hydrate Form A

Approximately 1 g of Compound (I) Form B freebase disclosed in US 2021/0221818 (corresponding to WO2021/150723) was added to acetonitrile (approximately 10 mL) to form a solution. Oxalic acid (approximately 135 mg) was added, and the solution was stirred for approximately 3 days. The resultant crystals were filtered and washed with acetonitrile (approximately 15 mL) and dried under vacuum at RT to provide 627 mg of Compound (I) oxalate crystalline salt Form A as a hydrate at 99.7% purity of the E-isomer (55% yield).

### 6.1 XRPD of Compound (I) Oxalate Crystalline Salt Hydrate Form A.

XRPD diffractograms of Compound (I) oxalate crystalline salt hydrate Form A were obtained with the instrumentation and procedures described above in Instrumentation Section (Option A). XPRD results were obtained as shown in FIG. 16 and FIG. 17. Peaks identified in FIGS. 16 and 17 include those listed in Tables 6A and 6B, below; optionally, the peaks reported in Table 6B may be considered as representative.

TABLE 6A
XRPD Data for Example 6 (Compound
(I) Oxalate Salt Hydrate Form A)

| Pos. [°2Th.] | Height [cts] | d-spacing [Å] | Rel. Int. [%] |
|---|---|---|---|
| 4.8032 | 2143.56 | 18.39795 | 41.38 |
| 9.3276 | 3189.81 | 9.48161 | 61.58 |
| 9.5140 | 2217.79 | 9.29628 | 42.81 |
| 11.0266 | 2235.53 | 8.02417 | 43.16 |
| 13.9852 | 2631.09 | 6.33259 | 50.79 |
| 14.1670 | 2587.30 | 6.25175 | 49.95 |
| 15.5804 | 670.61 | 5.68764 | 12.95 |
| 17.0073 | 3235.25 | 5.21352 | 62.45 |
| 17.9527 | 2320.57 | 4.94105 | 44.80 |
| 18.6866 | 5180.16 | 4.74862 | 100.00 |
| 19.5534 | 4662.80 | 4.54004 | 90.01 |
| 22.1479 | 2187.14 | 4.01371 | 42.22 |
| 22.6234 | 3805.00 | 3.93041 | 73.45 |
| 23.2344 | 1543.15 | 3.82842 | 29.79 |
| 26.4468 | 2065.79 | 3.37024 | 39.88 |
| 28.2146 | 1207.67 | 3.16296 | 23.31 |
| 29.9486 | 1024.30 | 2.98367 | 19.77 |
| 30.6226 | 417.23 | 2.91952 | 8.05 |
| 31.7225 | 1132.99 | 2.82076 | 21.87 |

TABLE 6B
Optional XRPD Data for Example 6 (Compound
(I) Oxalate Salt Hydrate Form A)

| Pos. [°2Th.] | Height [cts] | d-spacing [Å] | Rel. Int. [%] |
|---|---|---|---|
| 4.8032 | 2143.56 | 18.39795 | 41.38 |
| 9.3276 | 3189.81 | 9.48161 | 61.58 |
| 13.9852 | 2631.09 | 6.33259 | 50.79 |
| 14.1670 | 2587.30 | 6.25175 | 49.95 |
| 17.0073 | 3235.25 | 5.21352 | 62.45 |
| 18.6866 | 5180.16 | 4.74862 | 100.00 |
| 19.5534 | 4662.80 | 4.54004 | 90.01 |
| 22.6234 | 3805.00 | 3.93041 | 73.45 |

### 6.2. TGA and DSC/TGA.

Additional data for the oxalate crystalline salt hydrate Form A of Compound (I) prepared as described herein was obtained using the instrumentation and procedures described above in the Instrumentation Section (Option A). The results obtained are reported in FIG. 18 (TGA) and FIG. 19 (DSC/TGA).

### Example 7—Compound (I) Maleate Crystalline Salt Form B·MeCN Solvate

Form B of Compound (I) disclosed in US 2021/0221818 (corresponding to WO2021/150723) (102.7 mg) and maleic acid counterion (17.9 mg, 1 eq.) were stirred in MeCN (1 mL) at RT. The resulting solution was seeded with the maleate salt hydrate of Example 4.2 (1 mg); seed persisted. The slurry was stirred at RT for 2 days. The solids were filtered and air-dried for 4 hr which generated a new crystalline form—Compound (I) maleate crystalline salt Form B·MeCN solvate, which was confirmed by XRPD analysis (100 mg; 80% yield).

Additional preparation and characterization of the maleate salt were performed as follows. Form B of Compound (I) disclosed in US 2021/0221818 (corresponding to WO2021/150723) (1.3 g) and maleic acid solid (1 eq.) were stirred in MeCN (13 mL) at RT. The resulting slurry was seeded with the Compound (I) maleate crystalline salt Form B·MeCN solvate prepared as described herein; seed persisted. The mixture was stirred at RT for 3 days. The solids were filtered and air-dried for 4 hr to yield the maleate crystalline salt Form B·MeCN solvate (1.44 g, 94% yield). The XRPD pattern for Compound (I) maleate salt crystal Form B·MeCN solvate is shown in FIG. 20. The Compound (I) maleate crystalline salt Form B is a MeCN solvate. The DSC and TGA thermograms for this Form are shown in FIG. 21. DSC showed an endotherm with onset at 132.2° C. HPLC purity was 99.5% of the E-isomer. Proton NMR indicated it was the mono-maleate salt containing MeCN (0.7 eq).

### Example 8—Compound (I) Maleate Crystalline Salt Form A

Form B of Compound (I) disclosed in US 2021/0221818 (corresponding to WO2021/150723) (1.50 mmol) was stirred in 10.0 mL (10 vol.) of acetonitrile at 20° C. Maleic acid (1.50 mmol, 1.00 eq.) was added to the suspension in a single addition. The resulting mixture was stirred for 72 hr at 20° C. Next, vacuum filtration was performed to remove the mother liquor. The resultant wet cake was then washed with acetonitrile (3×5 mL, 5 vol.). A solid material was collected and dried in vacuo at 25° C. for 24 hr. The maleic acid salt of Compound (I) was obtained as a white solid (1.17 mmol, 78% yield). This material was identified as maleate crystalline salt Form A.

### 8.1 XRPD of Example 8 (Maleate Crystalline Salt of Compound (I) Form A).

XRPD diffractograms of Example 8 were collected with the instrumentation and procedures set forth in the Instrumentation Section (Option B). The XRPD pattern for the maleate crystalline salt Form A of Example 8 obtained is shown in FIG. 22, as an overlay with the XRPD patterns of maleic acid and Compound (I) Form B disclosed in US 2021/0221818 (corresponding to WO2021/150723).

TABLE 7
XRPD Data for Example 8 (Compound (I) Maleate Salt Form A)

| Pos. | Height | d-spacing | Rel. Int. |
|---|---|---|---|
| [°2Th.] | [cts] | [Å] | [%] |
| 9.7465 | 1562.11 | 9.07496 | 17.54 |
| 14.856 | 1454.21 | 5.96329 | 16.32 |
| 17.5655 | 2512.19 | 5.04909 | 28.2 |
| 18.9759 | 3010.34 | 4.67689 | 33.79 |
| 19.5772 | 8908.41 | 4.53456 | 100 |
| 21.599 | 6909.03 | 4.11446 | 77.56 |
| 22.354 | 3830.81 | 3.97716 | 43 |
| 22.7954 | 1963.33 | 3.90114 | 22.04 |

8.2. TGA. Additional data for the maleate crystalline salt Form A of Compound (I) was obtained using the procedures described above in the Instrumentation Section. The results obtained are reported in FIG. 23 (TGA).

### Example 9—Comparative Solubility Analysis for HCl, Oxalate and Maleate Crystalline Salts of Compound (I)

The above Examples show procedures for obtaining novel crystalline salt forms of Compound (I), which have enhanced solubilities as compared with the amorphous and previously reported Form B of Compound (I) disclosed in US 2021/0221818 (corresponding to WO2021/150723). The higher solubility of the inventive crystalline salts described herein may be useful in developing sustained release or modified release formulations and/or formulations offering improved bioabsorption. This Example reports illustrative data regarding the comparative solubility of the crystals. HPLC chromatograms used in this Example were analyzed at 225 nm.

### 9.1. Calibration.

To obtain comparative solubility data, a calibration curve was first generated using a stock solution of Form B of Compound (I) disclosed in US 2021/0221818 (corresponding to WO2021/150723) (20.0 mg) in methanol (10 mL), prepared in a volumetric flask. Using the stock solution, a dilution series for the purpose of HPLC quantitation was generated according to the following Table 8.

TABLE 8
Dilution Series for Calibration Curve

| | | | | | | | |
|---|---|---|---|---|---|---|---|
| Stock (μL) | 10 | 25 | 50 | 100 | 250 | 500 | 1000 |
| MeOH (μL) | 990 | 975 | 950 | 900 | 750 | 500 | 0 |
| Form B of | 0.020 | 0.050 | 0.100 | 0.200 | 0.500 | 1.000 | 2.000 |
| Compound (I) | | | | | | | |
| (mg/mL) | | | | | | | |

The purity (% area) of the Form B disclosed in US 2021/0221818 (corresponding to WO2021/150723) was used to correct the area on HPLC by dividing the measured area by the average purity (% area) of the 1 and 2 mg/mL solutions. Using linear regression, a linear best-fit equation was derived that expressed Compound (I) Form B concentration in mg/mL as a function of the area recorded on HPLC.

### 9.2. Comparative Solubility in Milli-Q Purified Water.

The following five-step process was then used to compare the solubility in water purified by a Milli-Q reverse osmosis system of Compound (I) Form B disclosed in US 2021/0221818 (corresponding to WO2021/150723), the amorphous form of Compound (I), and the HCl, oxalate and maleate salts of Examples 3, 6 and 8. First, a quantity of test sample was placed in a 2-dram vial equipped with stir bar. For each of Compound (I) Form B disclosed in US 2021/0221818 (corresponding to WO2021/150723), amorphous Compound (I), and the HCl crystalline salt, 30 mg of test sample was used in this first step; for the oxalate salt, 60 mg of test sample was used; and for the maleate salt, about 100 mg or more of test sample was used. In the second step, Milli-Q water was added to the vial under stirring followed by measuring the pH of the resulting suspension/solution. For each of Form B disclosed in US 2021/0221818 (corresponding to WO2021/150723), amorphous Compound (I), and the HCl crystalline salt, a sufficient volume of Milli-Q water was added to dissolve the test sample; for example, for the HCl salt, 3 mL of Milli-Q water was added, and for the oxalate salt, 2 mL of Milli-Q water was added in this second step and for the maleate, a greater volume of Milli-Q water was added. . . . After 1 hr of stirring, samples were withdrawn in sufficient quantities to enable further filtration and characterization (for example, 500 μL samples were withdrawn for Form B disclosed in US 2021/0221818 (corresponding to WO2021/150723), amorphous, and the HCl crystalline salt, and 150 μL was obtained for the oxalate). In each case, the sample was filtered through a 0.2 μm filter. The resulting solution was appropriately diluted to fall within the calibrated concentration range on HPLC. The sampling was repeated in identical fashion after 3 hr and 24 hr. After the last sample was taken, the pH was recorded again. The dissolved sample was determined by converting the recorded area on HPLC to concentration in mg/mL using the established calibration equation.

### 9.3. Comparative Solubility in Aqueous Buffers.

The procedures described in Example 9.2 were adjusted to determine the comparative solubility of the test samples in aqueous buffers of pH 4.5 and 6.8. As in Section 9.2, in the first step, a test sample (30 mg of Form B disclosed in US 2021/0221818 (corresponding to WO2021/150723), amorphous, or HCl salt; 20 mg of the oxalate salt), was placed in a 2-dram vial equipped with stir bar. Next, the second step of this modified procedure involved adding a buffer (5 mL) to the vial under stirring followed by measuring the pH of the resulting suspension. In case the pH drifted by more than 0.1 unit from the target pH, 10 μL aliquots of 1M NaOH (aq.) were used to readjust the pH to the original value. After 1 hr of stirring, the sample was withdrawn (1000 μL of Form B disclosed in US 2021/0221818 (corresponding to WO2021/150723), amorphous, or the HCl salt; 150 μL for the oxalate salt). As above in 9.2, the samples were filtered through a 0.2 μm filter, and the resulting solution was appropriately diluted to fall within the calibrated concentration range on HPLC. The sampling was repeated in an identical fashion after 3 hr and 24 hr and after the last sample was taken, the pH was recorded again. The dissolved sample was determined by converting the recorded area on HPLC to concentration in mg/mL using the established calibration equation.

### 9.4 Solubility Data.

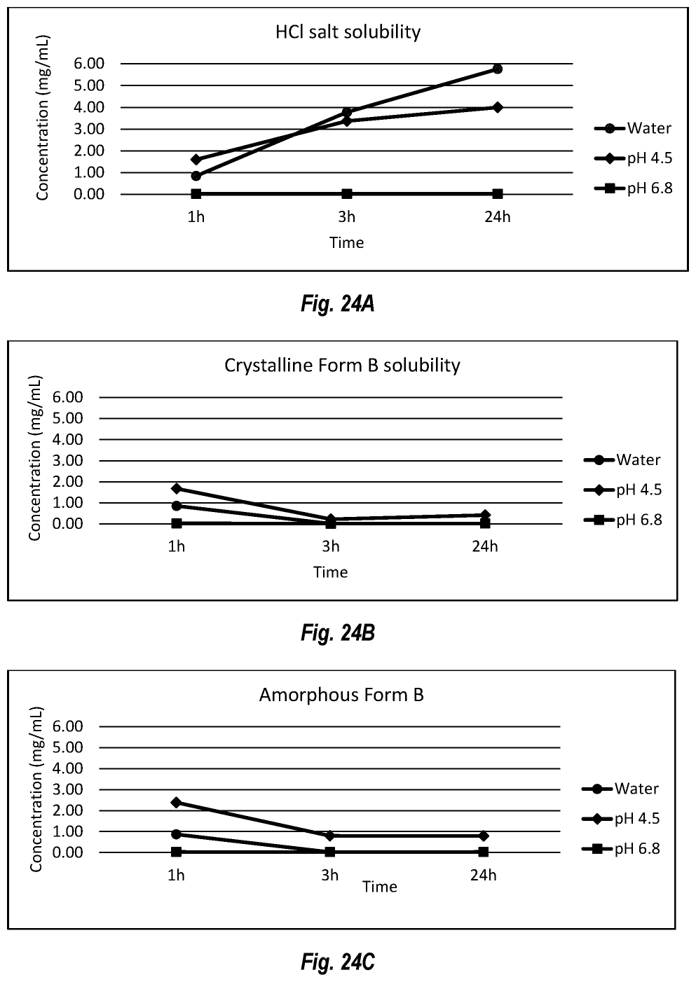

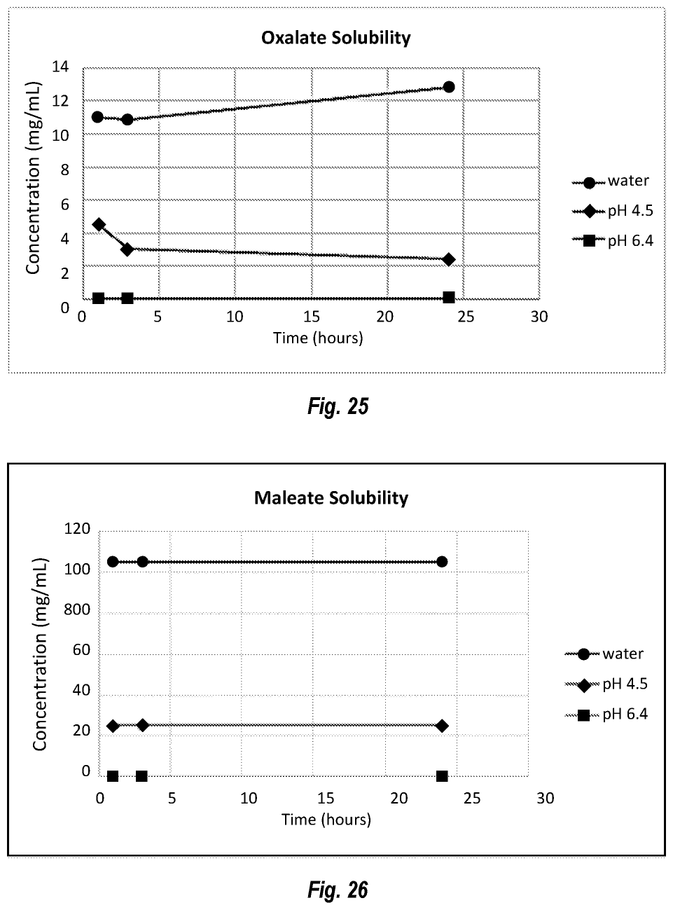

Applying the procedures of Example 9.1-9.3, solubility data was obtained as set forth in Tables 9-11. FIGS. 24A-C illustrate the data in graphic form demonstrating the significantly enhanced solubility of the HCl crystalline salt form of Compound (I) as compared with the amorphous and Compound (I) Form B disclosed in US 2021/0221818 (corresponding to WO2021/150723) solid forms. FIGS. 25 and 26 illustrate data in graphic form demonstrating the solubility of the oxalate and maleate crystalline salt forms of Compound (I), respectively, over time at variable pH.

Tables 9-11: Solubility Data for the crystalline salt forms of Examples 3, 6, and 8 as Compared with Compound (I) crystalline Form B and Compound (I) amorphous form Across Solvent Systems

TABLE 9
(In water)

| | | Solubility (mg/mL) | | | pH | |
|---|---|---|---|---|---|---|
| MEDIUM | SAMPLE | 1 hr | 3 hr | 24 hr | 0 hr | 24 hr |
| WATER | HCl salt | 0.9 | 3.8 | 5.8 | N/A | 4.0 |
| | Oxalate salt | 11.0 | 10.9 | 12.8 | N/A | 2.9 |
| | Maleate | >105 | >104 | >106 | N/A | 3.5 |
| | Compound (I) | 0.9 | 0.01 | 0.01 | N/A | 8.3 |
| | amorphous | | | | | |
| | Compound (I) | 0.9 | 0.01 | 0.01 | N/A | 7.8 |
| | Form B | | | | | |

TABLE 10
(In 50 mM Acetate)

| | | Solubility (mg/mL) | | | pH | |
|---|---|---|---|---|---|---|
| MEDIUM | SAMPLE | 1 hr | 3 hr | 24 hr | 0 hr | 24 hr |
| 50 mM | HCl salt | 1.6 | 3.4 | 4.0 | 4.5 | 4.4 |
| Acetate | Oxalate salt | 4.5 | 3.0 | 2.4 | 4.5 | 4.27 |
| | Maleate | >25 | >25 | >24 | 4.5 | 4.0 |
| | Compound (I) | 2.4 | 0.8 | 0.8 | 4.5 | 4.5 |
| | amorphous | | | | | |
| | Compound (I) | 1.7 | 0.3 | 0.4 | 4.5 | 4.5 |
| | Form B | | | | | |

TABLE 11
(In 100 mM Acetate)

| | | Solubility (mg/mL) | | | pH | |
|---|---|---|---|---|---|---|
| MEDIUM | SAMPLE | 1 hr | 3 hr | 24 hr | 0 hr | 24 hr |
| 100 mM | Oxalate salt | 2.6 | 2.7 | 2.3 | 4.5 | 4.5 |
| Acetate | Maleate | >16 | >16 | >16 | 4.5 | 4.5 |

### Example 10—Compound (I) Methyl Paraben Co-Crystal

Form B of Compound (I) disclosed in US 2021/0221818 (corresponding to WO2021/150723) (approximately 1 g) was weighed into a 100 mL round bottom flask, and methyl paraben (1.0 eq., 228.52 mg) was added to form a mixture. The sample and coformer (methyl paraben) were then dissolved in 10 vol. acetone (10 mL) aided with stirring. A clear solution formed to which 30 volumes of n-heptane (30 mL) was added, giving a light suspension. The suspension began to dissolve, and a further 20 vol (20 mL) of n-heptane was added. The sample remained a cloudy solution with a brown bilayer at the bottom of the flask. This was left to stir overnight. A white solid formed overnight which was filtered and air dried before being analysed by XRPD. The sample was confirmed to be the methyl paraben co-crystal of Compound (I). The material was allowed to dry in an oven under vacuum at 25° C. for 4 hr to provide 80.7% yield with 97.5% purity by HPLC. The DSC thermogram showed an onset at 133.5° C. with heat of enthalpy at 42 J/g; TGA thermogram showed weight loss of 2.6% between 25° C. and 250° C.

Upon further analysis, the methyl paraben co-crystal was found to be hygroscopic, absorbing 2.3% w/w of water between 0 and 90% RH. The material also remained the same XRPD pattern after GVS analysis, static storage conditions, grinding and compression studies, and after kinetic solubility analysis. Overall, the solid-state properties of methyl paraben co-crystalline form of Compound (I) are surprisingly positive as compared to Form B crystalline form of Compound (I) disclosed in US 2021/0221818 (corresponding to WO2021/150723), as shown by comparative grinding studies, compression studies, and static storage analyses set forth in Examples 11, 12 and 13 herein. The co-crystal is also readily prepared from anti-solvent addition and generates a good yield.

### 10.1. XRPD.

XRPD diffractograms were acquired with the instrumentation and procedures described in the Instrumentation Section (Option B). The XRPD pattern for the methyl paraben co-crystal is shown in FIG. 27. Peaks identified in FIG. 27 include those listed in Tables 12A and 12B, below, optionally, the peaks reported in Table 12B may be considered as representative.

TABLE 12A
XRPD Peaks for Example 10 (Methyl Paraben Co-Crystal)

| Intensity % | Angle | d value |
|---|---|---|
| 96 | 2-Theta ° | Angstrom |
| 93.2 | 4.6 | 19.068 |
| 19.9 | 9.3 | 9.488 |
| 18.7 | 13.1 | 6.751 |
| 30.1 | 14.4 | 6.131 |
| 20.0 | 15.4 | 5.735 |
| 40.8 | 16.6 | 5.346 |
| 32.8 | 17.7 | 5.010 |
| 55.4 | 18.3 | 4.837 |
| 100.0 | 19.3 | 4.584 |
| 33.1 | 20.2 | 4.401 |
| 25.7 | 21.0 | 4.229 |
| 33.5 | 21.6 | 4.114 |
| 49.8 | 22.5 | 3.941 |
| 24.3 | 25.5 | 3.486 |
| 20.3 | 26.0 | 3.418 |
| 19.7 | 26.9 | 3.314 |
| 20.7 | 27.4 | 3.249 |
| 18.3 | 28.0 | 3.187 |
| 16.1 | 29.2 | 3.053 |
| 39.4 | 10.8 | 8.183 |
| 27.4 | 11.2 | 7.922 |
| 15.4 | 14.0 | 6.314 |
| 20.3 | 16.2 | 5.482 |
| 16.6 | 16.9 | 5.232 |
| 25.7 | 17.3 | 5.134 |
| 32.0 | 22.2 | 3.997 |
| 20.0 | 23.3 | 3.811 |
| 26.0 | 23.7 | 3.754 |
| 22.9 | 23.9 | 3.714 |
| 14.3 | 25.3 | 3.517 |

TABLE 12B
XRPD Peaks for Example 10 (Methyl Paraben Co-Crystal)

| Intensity | Angle | d value |
|---|---|---|
| % | (°2θ) | Angstrom |
| 93.2 | 4.6 | 19.068 |
| 39.4 | 10.8 | 8.183 |
| 40.8 | 16.6 | 5.346 |
| 55.4 | 18.3 | 4.837 |
| 100 | 19.3 | 4.584 |
| 33.1 | 20.2 | 4.401 |
| 33.5 | 21.6 | 4.114 |
| 49.8 | 22.5 | 3.941 |

### 10.2. DSC/TGA.

Additional data for the methyl paraben co-crystalline salt form of Compound (I) was obtained using the procedures described above in the Instrumentation Section (Option B). The results obtained are reported in FIG. 28 (DSC/TGA thermograms).

### Example 11—Comparative Grinding Study for Compound (I) Form B and Compound (I): Methyl Paraben Co-Crystal

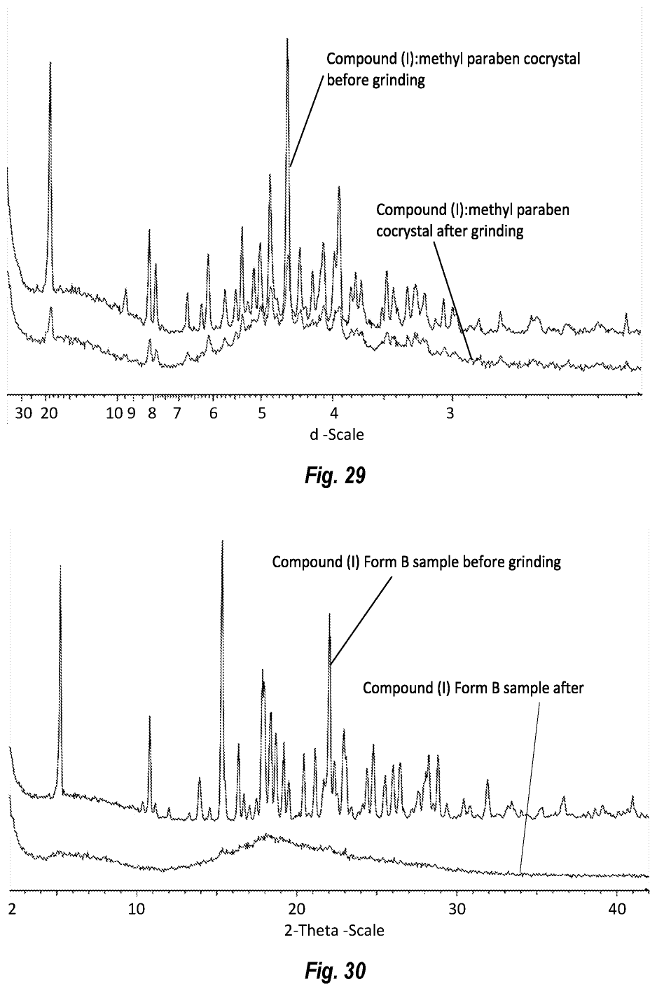

For the study, 40 mg of Form B crystalline form of Compound (I) disclosed in US 2021/0221818 (corresponding to WO2021/150723), and 40 mg of the methyl paraben co-crystal of Example 10, were weighed into separate steel containers, and a steel ball bearing equal in size was added to each of the containers. The samples were each ground at 30 Hz for 30 min, then reanalyzed by XRPD. XRPD analysis demonstrated that the methyl paraben co-crystal retained its co-crystalline structure pattern with only a limited reduction in overall crystallinity being observed, which can be seen in FIG. 29, which shows the XRPD patterns for methyl paraben pre- and post-grinding. In comparison, the sample comprising Form B crystalline form of Compound (I) disclosed in US 2021/0221818 (corresponding to WO2021/150723) became amorphous after grinding, which can be seen in FIG. 30 (which shows the XRPD patterns for Compound (I) Form B pre- and post-grinding).

### Example 12—Compression Study With Methyl Paraben Co-Crystal of Example 10

The methyl paraben co-crystalline form of Compound (I) after drying in a vacuum oven was compressed into a 6 mm recess disc under 100 kg of pressure for 2 min. The disc surface was analyzed by XRPD which produced the results shown in FIG. 31. As can be seen, after drying and compression, the methyl paraben co-crystal retained its co-crystalline structure pattern with only a limited reduction in overall crystallinity being observed.

### Example 13—Static Storage Analysis for Compound (I): Methyl Paraben Co-Crystal

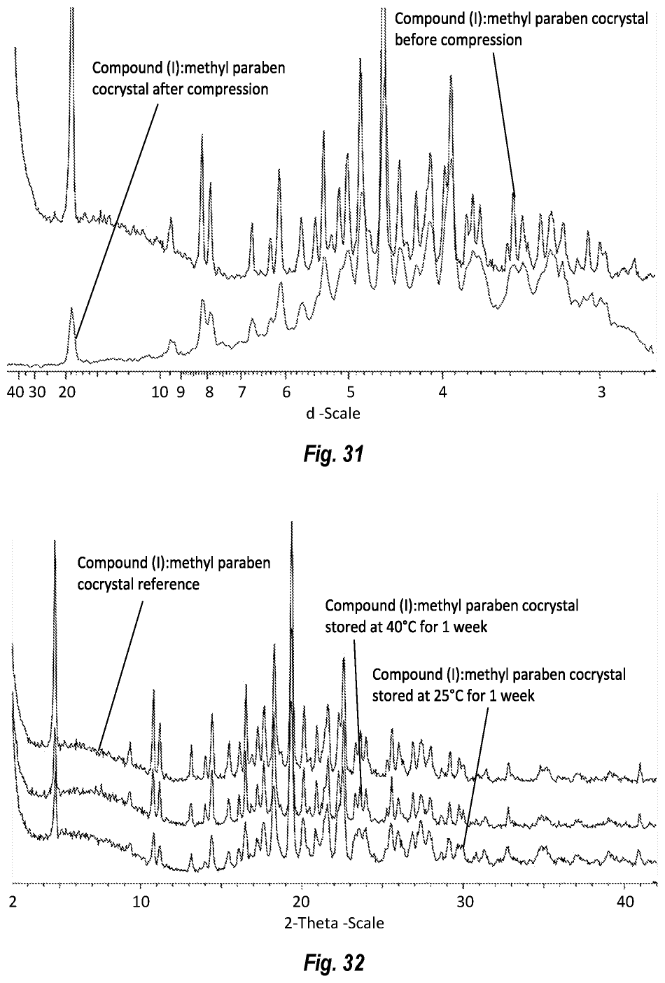

Samples of methyl paraben co-crystalline form of Compound (I) (Example 10), after drying in a vacuum oven were stored for one week at 25° C. and 40° C. After storage, XPRD analysis was conducted which produced the results in FIG. 32, demonstrating the methyl paraben co-crystalline form of Compound (I) retained its crystalline structure following storage and elevated temperature.

### Example 14—Solubility of Methyl Paraben Co-Crystal

The solubility of the methyl paraben co-crystal of Example 10 was also examined using a buffer solution at pH 4.5 and 6.8 at 1 hr, 3 hr and 24 hr time intervals. The sample was suspended in 1.5 mL media for a maximum anticipated concentration of Ca. 30 mg/mL of the free form of the Compound (I). The resulting suspensions were then shaken at 25° C./750 rpm for 24 hr. After 1 hr, 3 hr and 24 hr of equilibration, 0.5 mL of sample was aliquoted, the appearance was noted, and the pH of the saturated solution was measured. Samples were then centrifuged at 13,400 rpm for 2 min. Supernatant was used for HPLC analysis. Samples suspended in pH 4.5 buffer were diluted 1:1 (1- and 3-hr samples) and 1:8 (24 hr samples) with pH 4.5 buffer. Samples suspended in pH 6.8 buffer were analyzed undiluted. Quantitation was by HPLC with reference to a standard solution of approximately 0.15 mg/ml. . . . Different volumes of the standard diluted and undiluted sample solutions were injected. The solubility was calculated using the peak areas determined by integration of the peak found at the same retention time as the principal peak in the standard injection.

The methyl paraben co-crystal showed increased solubility over time at pH 4.5, starting at 0.65 mg/mL at the 1 hr time point and increasing to 3.9 mg/mL at the 24-hr time point. The overall solubility at pH 6.8 remained the same at 0.04 mg/mL at all three time points. The data obtained from this solubility assessment is shown below in Tables 13, 14, and 15. XRPD analysis reflected no change in the pattern post-solubility analysis (e.g., as to peaks set forth in Tables 12A and/or 12B).

TABLE 13
Methyl paraben co-crystal solubility 1-hour results

| | | | | Average |
|---|---|---|---|---|
| | | | Solubility | Solubility |
| Media | Appearance | pH | (mg/mL) | (mg/mL) |
| pH 4.5 | Suspension, Residual solid | 4.6 | 0.65 | 0.65 |
| buffer | Suspension, Residual solid | 4.6 | 0.64 | |
| pH 6.8 | Suspension, Residual solid | 6.8 | 0.04 | 0.04 |
| buffer | Suspension, Residual solid | 6.8 | 0.04 | |

TABLE 14
Methyl paraben co-crystal solubility 3-hour results

| | | | | Average |
|---|---|---|---|---|
| | | | Solubility | Solubility |
| Media | Appearance | pH | (mg/mL) | (mg/mL) |
| pH 4.5 | Suspension, Residual solid, solid at | 4.7 | 0.98 | 0.97 |
| buffer | side of vial | | | |
| | Suspension, Residual solid, solid at | 4.6 | 0.97 | |
| | side of vial | | | |
| pH 6.8 | Suspension, Residual solid | 6.8 | 0.04 | 0.04 |
| buffer | Suspension, Residual solid | 6.8 | 0.04 | |

TABLE 15
Methyl paraben co-crystal solubility 24-hour results

| | | | | Average |
|---|---|---|---|---|
| | | | Solubility | Solubility |
| Media | Appearance | pH | (mg/mL) | (mg/mL) |
| pH 4.5 | Milky suspension, residual solid | 4.7 | 4.0 | 3.9 |
| buffer | Milky suspension, residual solid | 4.7 | 3.7 | |
| pH 6.8 | Suspension, Residual solid | 6.8 | 0.04 | 0.04 |
| buffer | Suspension, Residual solid | 6.9 | 0.04 | |

The enhanced solubility of the crystalline forms of Compound (I) as shown in Examples 9 and 14 enable their use in formulating modified release and/or extended-release dosage forms of Compound (I).

### EQUIVALENTS

The foregoing written specification is considered sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and should be construed in accordance with the appended claims and any equivalents thereof.

What is claimed is:

1. A substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile chosen from an HCl salt, oxalate salt, or maleate salt form thereof.

2. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl birefringent crystalline salt.

3. The substantially crystalline form of claim 2, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl birefringent crystalline salt is characterized by an XRPD pattern substantially the same as those in FIG. 1

4. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt hydrate.

5. The substantially crystalline form of claim 4, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt hydrate is characterized by at least one of
an XRPD pattern substantially the same as FIG. 2;
a DSC/TGA profile substantially the same as FIG. 3.

6. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt Form A.

7. The substantially crystalline form of claim 6, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt Form A is characterized by at least one of
an XRPD pattern substantially the same as FIG. 4;
an XRPD pattern substantially the same as FIG. 5;
a DSC profile substantially the same as FIG. 6;
a TGA profile substantially the same as FIG. 7.

8. The substantially crystalline form of claim 6 or 7, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: HCl crystalline salt Form A is characterized by an XRPD pattern comprising four or more peaks, in terms of 2-theta degrees, chosen from peaks at about 9.7±0.2, 14.9±0.2, 17.6±0.2, 19.0±0.2, 19.6±0.2, 21.6±0.2, 22.3±0.2, and 29.3±0.3.

9. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate birefringent crystalline salt Form A.

10. The substantially crystalline form of claim 9, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate birefringent crystalline salt Form A is characterized by at least one of:
an XRPD pattern substantially the same as FIG. 8;
a DSC/TGA profile substantially the same as FIG. 9.

11. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate birefringent crystalline salt.

12. The substantially crystalline form of claim 11, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate birefringent crystalline salt is characterized by at least one of:
an XRPD pattern substantially the same as FIG. 10;
a DSC/TGA profile substantially the same as FIG. 11.

13. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form A.

14. The substantially crystalline form of claim 13, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form A is characterized by at least one of:
an XRPD pattern substantially the same as FIG. 14;
a DSC profile substantially the same as FIG. 15A;
an XRPD pattern substantially the same as FIG. 16;
an XRPD pattern substantially the same as FIG. 17;
a TGA profile substantially the same as FIG. 18;
a DCS/TGA profile substantially the same as FIG. 19.

15. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form B.

16. The substantially crystalline form of claim 15, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form B is characterized by at least one of:
an XRPD pattern substantially the same as FIG. 12;
a DSC/TGA profile substantially the same as FIG. 13;
a DSC profile substantially the same as FIG. 15B.

17. The substantially crystalline form of claim 13 or 14, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: oxalate crystalline salt hydrate Form A is characterized by an XRPD pattern comprising four or more peaks, in terms of 2-theta degrees, chosen from peaks at about 4.8±0.2, 9.3±0.2, 14.0±0.2, 14.2±0.2, 17.0±0.2, 18.7±0.2, 19.6±0.2, and 22.6±0.2.

18. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form B·MeCN solvate.

19. The substantially crystalline form of claim 18, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form B·MeCN solvate is characterized by at least one of:
an XRPD pattern substantially the same as FIG. 20;
a DSC/TGA profile substantially the same as FIG. 21.

20. The substantially crystalline form of claim 1, which is 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form A.

21. The substantially crystalline form of claim 20, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form A is characterized by at least one of:
an XRPD pattern substantially the same as shown in FIG. 22;
a TGA profile substantially the same as FIG. 23.

22. The substantially crystalline form of claim 20 or 21, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: maleate crystalline salt Form A is characterized by an XRPD pattern comprising four or more peaks, in terms of 2-theta degrees, chosen from peaks at about 9.7±0.2, 14.9±0.2, 17.6±0.2, 19.0±0.2, 19.6±0.2, 21.6±0.2, 22.3±0.2, and 22.8±0.2.

23. A substantially crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, which is a methyl paraben co-crystal.

24. The substantially crystalline form of claim 23, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: methyl paraben co-crystal is characterized by at least one of:
an XRPD pattern substantially the same as FIG. 27;
a DCS/TGA profile substantially the same as FIG. 28.

25. The substantially crystalline form of claim 23 or 24, wherein the 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile: methyl paraben co-crystal is characterized by an XRPD pattern comprising four or more peaks, in terms of 2-theta degrees, chosen from peaks at about 4.6±0.2, 10.8±0.2, 16.6±0.2, 18.3±0.2, 19.3±0.2, 20.2±0.2, 21.6±0.2 and 22.5±0.2.

26. The substantially crystalline form of any one of claims 1 to 25, which is at least 50% crystalline form, such as at least 60%, at least 70%, at least 80%, at least 90%, at least 95%, at least 96%, at least 97%, at least 98%, at least 99%, or 100% crystalline.

27. A pharmaceutical composition comprising at least one substantially crystalline form of any of the preceding claims and a pharmaceutically acceptable excipient.

28. A method of treating a disease mediated by activity of BTK in a subject in need of treatment comprising administering to the subject a crystalline form of 2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, according to any one of claims 1 to 26, or pharmaceutical composition according to claim 27.
